SEQUENCE LISTING (16 sequences)

SEQ ID NO: 1 - protein, 3 aa, Artificial Sequence
  targeting ligand
RGD

SEQ ID NO: 2 - protein, 7 aa, Artificial Sequence
  Targeting ligand
GRGDSPK

SEQ ID NO: 3 - protein, 5 aa, Artificial Sequence
  Targeting ligand
GRGDS

SEQ ID NO: 4 - protein, 6 aa, Artificial Sequence
  Targeting ligand
GRGDSP

SEQ ID NO: 5 - protein, 4 aa, Artificial Sequence
  Targeting ligand
RGDS

SEQ ID NO: 6 - protein, 10 aa, Artificial Sequence
  Targeting ligand
RGDSPASSKP

SEQ ID NO: 7 - protein, 5 aa, Artificial Sequence
  RGDFV
  MISC_FEATURE (4)..(4): Phe is d-Phe
RGDFV

SEQ ID NO: 8 - protein, 5 aa, Artificial Sequence
  Targeting ligand
  MISC_FEATURE (4)..(4): Phe is d-Phe
RGDFC

SEQ ID NO: 9 - protein, 5 aa, Artificial Sequence
  Targeting sequence
  MISC_FEATURE (4)..(4): Phe is d-Phe
RGDFE

SEQ ID NO: 10 - protein, 5 aa, Artificial Sequence
  Targeting sequence
  MISC_FEATURE (4)..(4): Phe is d-Phe
RGDFK

SEQ ID NO: 11 - protein, 5 aa, Artificial Sequence
  Targeting sequence
  MISC_FEATURE (4)..(4): Tyr is d-Tyr
RGDYC

SEQ ID NO: 12 - protein, 5 aa, Artificial Sequence
  Targeting sequence
  MISC_FEATURE (4)..(4): Tyr is d-Tyr
RGDYE

SEQ ID NO: 13 - protein, 5 aa, Artificial Sequence
  Targeting sequence
  MISC_FEATURE (4)..(4): Tyr is d-Tyr
RGDYK

SEQ ID NO: 14 - protein, 5 aa, Artificial Sequence
  Targeting sequence
  MISC_FEATURE (4)..(4): Tyr is d-Try
RGDYV

SEQ ID NO: 15 - protein, 5 aa, Artificial Sequence
  Targeting sequence
  MISC_FEATURE: 3-Aminomethylbenzoyl
  misc_feature (5)..(5): Xaa can be any naturally occurring amino acid
ARGDX

SEQ ID NO: 16 - protein, 10 aa, Artificial Sequence
  Targeting sequence
CRGDSPASSC

(19) United States
(12) Patent Application Publication
(10) Pub. No.: US 2023/0125052 A1
(43) Pub. Date: Apr. 20, 2023

(54) RED BLOOD CELL-DERIVED VESICLE

(71) Applicant: Imperial College Innovations Limited, London (GB)

(72) Inventors: Rongjun CHEN, London (GB); Yu HUANG, London (GB); Xiao Yun XU, London (GB); Simon THOM, London (GB)

(73) Assignee: Imperial College Innovations Limited, London (GB)

(21) Appl. No.: 17/786,403

(22) PCT Filed: Dec. 18, 2020
(86) PCT No.: PCT/GB2020/053276

§ 371 Date: Jun. 16, 2022

(30) Foreign Application Priority Data
Dec. 20, 2019 (GB) 1919075.0

(51) Int. Cl. A61K 9/50; A61K 38/48; A61K 31/616; A61P 7/02

(52) U.S. Cl. CPC A61K 9/5068; A61K 9/5089; A61K 31/616; A61K 38/482; A61P 7/02; C12Y 304/21068

(57) ABSTRACT

The invention relates to red blood cell-derived vesicles comprising encapsulated active agents, their use in therapy and methods of production thereof.

The invention relates to red blood cell-derived vesicles, their use in therapy and methods of production thereof.

Vascular thrombosis is a major clinical problem. It accounts for about half of all deaths particularly in developed Western countries as a result of catastrophic thrombotic diseases, such as ischemic stroke, myocardial infarction and pulmonary embolism. Timely lysis and/or removal of blood clots to rapidly re-establish blood flow is critical to treat those thrombotic diseases.

One of the currently available clinical strategies is intravenous infusion of a fibrinolytic agent, such as the most widely used tissue plasminogen activator (tPA), which converts plasminogen to plasmin and thus triggers fibrin lysis. Unfortunately, tPA has an extremely short half-life (2~6 min); it is rapidly inactivated by circulating inhibitors such as plasminogen activator inhibitor-1 (PAI-1). Thus, large doses are required for effective thrombolysis. However, excessive administration and systemic distribution of tPA are detrimental because it impairs normal haemostatic capabilities and leads to bleeding complications. In the past two decades, extensive studies have been focused on exploring alternative thrombolytic agents to tPA, and most drugs have failed in clinical trials.

Although efforts have been reported to improve the stability of tPA in the circulation through PEGylation and/or nanoencapsulation, it remains a major challenge to achieve selective accumulation of tPA at a clot-site to enable targeted thrombolysis. Covalent conjugation of fibrin-specific antibody to tPA has been used, but direct conjugation has been reported to result in reduced biological activity (Proc. Natl. Acad. Sci. USA 1987, 84, 7659). Korin et al. has reported poly(lactic-co-glycolic acid)-based, shear-active nanotherapeutics, by taking advantage of the high shear stress caused by arterial narrowing for targeted delivery of tPA to obstructed blood vessels (Science 2012, 337, 738). Another targeted system has been developed by utilising magnetically activated nanomotors to improve the transport of tPA at the blood clot surface for more effective local ischemic stroke therapy (ACS Nano 2014, 8, 7746). Nevertheless, the dependence on luminal high shear stress or an external rotating magnetic nanomotor makes it difficult to apply such systems in a general clinical setting. In addition, applications of magnetically controlled systems are limited due to absorption and/or possible damage to normal tissues.

So far, researchers have to rely on the conventional PEGylation method to improve the stability and prolong blood circulation of thrombolytics, such as tPA (Biomaterials 2009, 30, 5751; Microb Cell Fact. 2017, 16, 197; Mol. Med. Rep. 2017, 16, 4909). However, other researchers have reported that the polyethylene glycol (PEG) moiety in itself may be immunogenic and that the induced anti-PEG antibodies are linked to enhanced blood clearance and reduced efficacy (Pharm. Res. 2013, 30, 1729; Expert Opin. Drug Deliv. 2012, 9, 1319; J Control Release. 2007, 119, 236).

There is therefore a considerable clinical need for a targeted thrombolytic drug delivery system, which enables the drug to persist in the circulation and achieve selective thrombolysis without increasing off-target bleeding.

According to a first aspect, there is provided a red blood cell-derived vesicle (RBCV) comprising an encapsulated active agent.

Advantageously, the RBCVs of the invention result in superior in vivo residence time when compared to conventional PEG coating in liposomal systems. The abundant “self-markers” including immunosuppressive proteins present on the surface of natural RBCs collected from the host blood (for autologous therapy) or from the donor blood (for allogeneic therapy) can display significantly reduced macrophage uptake and negligible immune response. This is important since researchers have reported that the PEG moiety in itself may be immunogenic and that the induced anti-PEG antibodies are linked to enhanced blood clearance and reduced efficacy. Thus, the abundant “self-markers” including proteins, glycan, and sialic acid moieties present on the RBC membrane can play a critical role in suppressing immune attack, which results in a very long circulation (about 120 days) of red blood cells in man.

The encapsulated active agent is preferably a thrombolytic agent. The skilled person would appreciate that a thrombolytic agent is an agent that is capable of dissolving a blood clot (thrombus) and reopening an artery or vein. Thus, the active agent is preferably configured for blood clot lysis.

The thrombolytic agent may be selected from a group consisting of: a fibrinolytic agent; a von Willebrand factor-cleaving protease (VWFCP); and a DNase that is capable of degrading neutrophil extracellular traps (NETs).

The fibrinolytic agent may be tissue plasminogen activator (tPA), tenecteplase (TNK), reteplase, urokinase (UK), streptokinase (SK), anistreplase (also known as anisoylated plasminogen streptokinase activator complex (APSAC)), or lumbrokinase.

The von Willebrand factor-cleaving protease (VWFCP) may be a disintegrin, a metalloproteinase with a thrombospondin type 1 motif or member 13 (ADAMTS13).

Thus, the thrombolytic agent may be selected from a group consisting of: fibrinolytics including tissue plasminogen activator (tPA), tenecteplase (TNK), reteplase, urokinase (UK), streptokinase (SK), anistreplase (also known as anisoylated plasminogen streptokinase activator complex (APSAC)), and lumbrokinase; von Willebrand factor-cleaving protease (VWFCP) including a disintegrin and metalloproteinase with a thrombospondin type 1 motif, member 13 (ADAMTS13); and a DNase that is capable of degrading neutrophil extracellular traps (NETs).

Preferably, the thrombolytic agent is tPA or tenecteplase. Most preferably, however, the thrombolytic agent is tPA.

In an alternative embodiment of the invention the active agent may be an anti-platelet agent.

The anti-platelet agent may be selected from a group consisting of: GPIIb-IIIa (α_{IIb}β₃) inhibitors; irreversible cyclooxygenase inhibitors; adenosine diphosphate (ADP) receptor inhibitors; adenosine reuptake inhibitors; phosphodiesterase inhibitors; protease-activated receptor-1 (PAR-1) antagonists; and thromboxane inhibitors.

The GPIIb-IIIa (α_{IIb}β₃) inhibitor may be eptifibatide, abciximab or tirofiban.

The irreversible cyclooxygenase inhibitor may be aspirin or triflusal.

The adenosine diphosphate (ADP) receptor inhibitor may be clopidogrel, cangrelor, prasugrel, ticagrelor or ticlopidine.

The adenosine reuptake inhibitor may be dipyridamole.

The phosphodiesterase inhibitor may be cilostazol.

The protease-activated receptor-1 (PAR-1) antagonist may be vorapaxar.

The thromboxane inhibitors may be a thromboxane synthase inhibitor or a thromboxane receptor antagonist.

Thus, the anti-platelet agent may be selected from a group consisting of: GPIIb-IIIa (α_{IIb}β₃) inhibitors including eptifibatide, abciximab and tirofiban; irreversible cyclooxygenase inhibitors including aspirin and triflusal; adenosine diphosphate (ADP) receptor inhibitors including clopidogrel, cangrelor, prasugrel, ticagrelor, ticlopidine; adenosine reuptake inhibitors including dipyridamole; phosphodiesterase inhibitors including cilostazol; protease-activated receptor-1 (PAR-1) antagonists including vorapaxar; and thromboxane inhibitors including thromboxane synthase inhibitors and thromboxane receptor antagonists.

Preferably, the anti-platelet agent is an irreversible cyclooxygenase inhibitor. Most preferably, the anti-platelet agent is acetylsalicylic acid (aspirin, ASA).

The RBCV may comprise more than one active agent. For example, the RBCV may encapsulate tPA and ADAMTS₁₃; or tPA and DNase; or tPA, ADAMTS₁₃ and DNase; or tPA and tenecteplase; or tPA and streptokinase; or reteplase and urokinase; or tPA and aspirin; or tPA, ADAMTS₁₃ and aspirin; or tPA, DNase and aspirin; or tPA, ADAMTS₁₃, DNase and aspirin; or aspirin and dipyridamole; or aspirin and clopidogrel, or any combination or two or more of the active agents mentioned herein.

In an embodiment, the red blood cell-derived vesicle (RBCV) may have an average diameter of between 1 nm and 1000 nm, or between 10 nm and 1000 nm, or between 100 nm and 1000 nm, or between 200 nm and 1000 nm, or between 300 nm and 1000 nm, or between 400 nm and 1000 nm, or between 500 nm and 1000 nm.

In another embodiment, the RBCV may have an average diameter of between 100 nm and 900 nm, between 200 nm and 900 nm, between 300 nm and 900 nm, between 400 nm and 900 nm, between 500 nm and 900 nm. Alternatively, the RBCV may have an average diameter of between 100 nm and 800 nm, between 200 nm and 800 nm, between 300 nm and 800 nm, between 400 nm and 800 nm, between 500 nm and 800 nm. Alternatively, the RBCV may have an average diameter of between 100 nm and 700 nm, between 200 nm and 700 nm, between 300 nm and 700 nm, between 400 nm and 700 nm, between 500 nm and 700 nm. Alternatively, the RBCV may have an average diameter of between 100 nm and 600 nm, between 200 nm and 600 nm, between 300 nm and 600 nm, between 400 nm and 600 nm, between 500 nm and 600 nm.

Preferably, however, the average diameter of the red blood cell-derived vesicle is between 100 nm and 1000 nm. Most preferably, the average diameter of the red blood cell-derived vesicle is between 200 nm and 500 nm. The diameter of the vesicle may be measured using dynamic light scattering, as shown in FIG. 3, and/or transmission electron microscopy, as shown in FIG. 4 and described in the materials and methods section below.

The red blood cell vesicle may comprise a zeta potential of between -1 mV and -100 mV, preferably between -10 mV and -50 mV, more preferably between -20 mV and -40 mV, and most preferably between -25 mV and -35 mV. The zeta potential of the vesicle may be determined using a zeta potential analyser, as shown in the examples and described in the materials and methods section below.

The red blood cell-derived vesicle may further comprise at least one targeting ligand (or targeting moiety) attached to the surface thereto. Preferably, the vesicle is functionalised (or decorated) with a plurality of spaced apart targeting ligands or moieties. Preferably, the at least one targeting ligand is configured for thrombus-targeted delivery of the active agent.

The at least one targeting ligand (or targeting moiety) may be configured to selectively target the vesicle to a cell involved in blot clotting or other factors present in the characteristic microenvironment of a thrombus site.

Preferably, the targeting ligand (or targeting moiety) may be configured to selectively target the vesicle to a factor present in the characteristic microenvironment of a thrombus site. Preferably, the factor may be selected from the group consisting of: glycoprotein; fibrin; thrombin; collagen and P-selectin.

The targeting ligand (or targeting moiety) may be a cell targeting ligand. Thus, preferably, the targeting ligand may be configured to selectively target the vesicle to a cell involved in blot clotting. Preferably, the at least one targeting ligand is configured to selectively target the active agent to a platelet, preferably an activated platelet.

The term “activated platelet” can refer to activation of a platelet when endothelial damage occurs, and the platelets come into contact with exposed collagen and von Willebrand factor, thereby becoming “activated”. Platelets may also be activated by thrombin or by a negatively charged surface, such as glass. Platelet activation may result in the scramblase-mediated transport of negatively charged phospholipids to the platelet surface, providing a catalytic surface for the tenase and prothrombinase complexes. Activated platelets may be characterised by a change in morphology and pseudopods forming on their surface, when compared to non-activated platelets, defining a star-like appearance.

Preferably, the at least one targeting ligand (or targeting moiety) binds to a receptor. Preferably, the receptor may be selected from a group consisting of: active integrin GPIIb-IIIa (α_{IIb}β₃); P-selectin; a GPIb-type receptor; thrombospondin; a C-X-C type chemokine receptor; and a thrombin receptor. In an embodiment of the invention, the at least one targeting ligand (or targeting moiety) binds to the same species or type of receptors, or to two or more different types or species of receptors.

Most preferably, however, the at least one targeting ligand (or targeting moiety) binds to α_{IIb}β₃ integrin.

Thus, the red blood cell-derived vesicle comprising one or more cell targeting ligand (or targeting moiety) attached to the surface thereto may act to mimic fibrinogen.

The skilled person would understand that fibrinogen is capable of binding to activated platelets through the arginine-glycine-aspartic acid (RGD) motifs located on the two Aα chains, of GPIIb-IIIa (α_{IIb}β₃) integrin, which leads to platelet aggregation through the “bridging effect”. Thus, preferably the red blood cell-derived vesicle comprising one or more cell targeting ligand mimics fibrinogen binding to activated platelets.

The at least one targeting ligand (or targeting moiety) may be an antibody, or an antigen binding fragment thereof, and more preferably an antibody, or an antigen binding fragment thereof that is capable of binding to an epitope displayed on a cell involved in blot clotting or a factor present in the characteristic microenvironment of a thrombus site.

Preferably, the antibody, or antigen binding fragment thereof is capable of binding to a factor selected from the group consisting of: thrombin; fibrin; collagen; glycoprotein and P-selectin.

Preferably, the antibody, or antigen binding fragment thereof is capable of binding to an epitope displayed on the cell surface of a platelet, most preferably an activated platelet.

The term “epitope” can mean any region of an antigen with the ability to elicit, and combine with, a binding region of the antibody or antigen-binding fragment thereof.

The antibody may be monoclonal or polyclonal. Preferably, the antibody is a monoclonal antibody.

The term “antigen binding fragment” of an antibody can mean a portion of the antibody which retains a functional activity. A functional activity can be, for example antigen binding activity or specificity. A functional activity can also be, for example, an effector function provided by an antibody constant region. The term “antigen binding fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab‘; bivalent fragments such as F(ab’)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-₁. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab’ fragment” can mean a monovalent antigen-binding fragment of a human monoclonal antibody that is larger than a Fab fragment. For example, a Fab’ fragment includes all of the light chain, all of the variable region of the heavy chain, and all or part of the first and second constant domains of the heavy chain. For example, a Fab’ fragment can additionally include some or all of amino acid residues 220 to 330 of the heavy chain.

The term “F(ab’)2 fragment” can mean a bivalent antigen-binding fragment of a human monoclonal antibody. An F(ab’)₂ fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

The antigen-binding fragment thereof may comprise or consist of any of the fragments selected from a group consisting of VH, VL, Fd, Fv, Fab, Fab’, scFv, F(ab’)₂ and Fc fragment.

Preferably, the antibody, or an antigen binding fragment is capable of binding to an integrin. Most preferably the antibody, or an antigen binding fragment is capable of binding to aIIbβ₃ integrin.

The at least one targeting ligand (or targeting moiety) may be a cyclic or linear peptide.

Preferably, the at least one targeting ligand (or targeting moiety) is a linear peptide. In one embodiment, the at least one targeting ligand (or targeting moiety) may be a peptide selected from a group of peptides consisting of:
Arg-Gly-Asp - SEQ ID No: 1;
Gly-Arg-Gly-Asp-Ser-Pro-Lys - SEQ ID No: 2;
Gly-Arg-Gly-Asp-Ser - SEQ ID No: 3;
Gly-Arg-Gly-Asp-Ser-Pro (GRGDSP) - SEQ ID No: 4;
Arg-Gly-Asp-Ser - SEQ ID No: 5; and
Arg-Gly-Asp-Ser-Pro-Ala-Ser-Ser-Lys-Pro - SEQ ID No: 6.

Preferably, the peptide is Arg-Gly-Asp - SEQ ID No: 1, which may also be referred to as Arginylglycylaspartic acid (RGD).

Preferably, the at least one targeting ligand (or targeting moiety) is a cyclic peptide. In one embodiment, the at least one targeting ligand (or targeting moiety) may be a peptide selected from a group of peptides consisting of:
Cyclo (Arg-Gly-Asp-d-Phe-Val) - SEQ ID No: 7;
Cyclo (Arg-Gly-Asp-d-Phe-Cys) - SEQ ID No: 8;
Cyclo (Arg-Gly-Asp-d-Phe-Glu) - SEQ ID No: 9;
Cyclo (Arg-Gly-Asp-d-Phe-Lys) - SEQ ID No: 10;
Cyclo (Arg-Gly-Asp-d-Tyr-Cys) - SEQ ID No: 11;
Cyclo (Arg-Gly-Asp-d-Tyr-Glu) - SEQ ID No: 12;
Cyclo (Arg-Gly-Asp-d-Tyr-Lys) - SEQ ID No: 13;
Cyclo (Arg-Gly-Asp-d-Tyr-Val) - SEQ ID No: 14;
Cyclo (Ala-Arg-Gly-Asp-3-Aminomethylbenzoyl) - SEQ ID No: 15; and
Cyclo(Cys-Arg-Gly-Asp-Ser-Pro-Ala-Ser-Ser-Cys) - SEQ ID No: 16.

Preferably, the peptide is cyclo (Arg-Gly-Asp-d-Phe-Val) - SEQ ID No: 7.

Preferably, the at least one targeting ligand (or targeting moiety) may be a linear RGD-or cyclic RGD (cRGD)-containing peptide.

Preferably, the at least one targeting ligand (or targeting moiety) is present on the surface of the red blood cell-derived vesicle at a density of between about 1 and 10,000 ,000 per vesicle, between about 10 and 10,000 ,000 per vesicle, between about 50 and 10,000 ,000 per vesicle, between about 100 and 10,000 ,000 per vesicle, between about 1000 and 10,000 ,000 per vesicle, between about 10,000 and 10,000 ,000 per vesicle, between about 100,000 and 10,000 ,000 per vesicle, or between about 1,000,000 and 10,000 ,000 per vesicle, or between about 5,000,000 and 10,000 ,000 per vesicle.

Preferably, the at least one targeting ligand (or targeting moiety) is present on the surface of the red blood cell-derived vesicle at a density of between about 1 and 1,000 ,000 per vesicle, between about 10 and 1,000 ,000 per vesicle, between about 50 and 1,000 ,000 per vesicle, between about 100 and 1,000 ,000 per vesicle, between about 1000 and 1,000 ,000 per vesicle, between about 10,000 and 1,000 ,000 per vesicle, or between about 100,000 and 1,000 ,000 per vesicle.

Preferably, the at least one targeting ligand (or targeting moiety) is present on the surface of the red blood cell-derived vesicle at a density of between about 1 and 100,000 per vesicle, between about 10 and 100,000 per vesicle, between about 50 and 100,000 per vesicle, between about 100 and 100,000 per vesicle, between about 1000 and 100,000 per vesicle, or between about 10,000 and 100,000 per vesicle.

Preferably, the at least one targeting ligand (or targeting moiety) is present on the surface of the red blood cell-derived vesicle at a density of between about 1 and 10,000 per vesicle, between about 10 and 10,000 per vesicle, between about 50 and 10,000 per vesicle, between about 100 and 10,000 per vesicle, or between about 1000 and 10,000 per vesicle.

Preferably, the at least one targeting ligand (or targeting moiety) is present on the surface of the red blood cell-derived vesicle at a density of between about 1 and 1000 per vesicle, between about 10 and 1000 per vesicle, between about 50 and 1000 per vesicle, or between about 100 and 1000 per vesicle.

Preferably, the at least one targeting ligand (or targeting moiety) is present on the surface of the red blood cell-derived vesicle at a density of between about 200 and 1000 per vesicle, between about 300 and 1000 per vesicle, between about 400 and 1000 per vesicle, between about 500 and 1000 per vesicle, between about 600 and 1000 per vesicle, between about 700 and 1000 per vesicle, between about 800 and 1000 per vesicle, or between about 900 and 1000 per vesicle.

Preferably, the at least one targeting ligand (or targeting moiety) is present on the surface of the red blood cell-derived vesicle at a density of between about 10 and 500 per vesicle, between about 10 and 400 per vesicle, between about 10 and 300 per vesicle, between about 10 and 200 per vesicle, or between about 10 and 150 per vesicle.

Preferably, the at least one targeting ligand (or targeting moiety) is present on the surface of the red blood cell-derived vesicle at a density of between about 50 and 500 per vesicle, between about 50 and 400 per vesicle, between about 50 and 300 per vesicle, between about 50 and 200 per vesicle, or between about 50 and 150 per vesicle.

Preferably, the at least one targeting ligand (or targeting moiety) is present on the surface of the red blood cell-derived vesicle at a density of between about 50 and 150 per vesicle.

Most preferably, the at least one targeting ligand (or targeting moiety) is present on the surface of the red blood cell-derived vesicle at a density of at least about 50 per vesicle.

Most preferably, the at least one targeting ligand (or targeting moiety) is present on the surface of the red blood cell-derived vesicle at a density of at least about 100 per vesicle.

Most preferably, the at least one targeting ligand (or targeting moiety) is present on the surface of the red blood cell-derived vesicle at a density of at least about 500 per vesicle.

Most preferably, the at least one targeting ligand (or targeting moiety) is present on the surface of the red blood cell-derived vesicle at a density of at least about 1000 per vesicle.

Most preferably, the at least one targeting ligand (or targeting moiety) is present on the surface of the red blood cell-derived vesicle at a density of at least about 10,000 per vesicle.

Most preferably, the at least one targeting ligand (or targeting moiety) is present on the surface of the red blood cell-derived vesicle at a density of at least about 100,000 per vesicle.

Most preferably, the at least one targeting ligand (or targeting moiety) is present on the surface of the red blood cell-derived vesicle at a density of at least about 1,000,000 per vesicle.

Most preferably, the at least one targeting ligand (or targeting moiety) is present on the surface of the red blood cell-derived vesicle at a density of at least about 10,000,000 per vesicle.

Most preferably, the at least one targeting ligand (or targeting moiety) is present on the surface of the red blood cell-derived vesicle at a density of about 100 per vesicle.

Most preferably, the at least one targeting ligand (or targeting moiety) is present on the surface of the red blood cell-derived vesicle at a density of about 1000 per vesicle.

Most preferably, the at least one targeting ligand (or targeting moiety) is present on the surface of the red blood cell-derived vesicle at a density of about 10,000 per vesicle.

Most preferably, the at least one targeting ligand (or targeting moiety) is present on the surface of the red blood cell-derived vesicle at a density of at least about 20,000 per vesicle.

Most preferably, the at least one targeting ligand (or targeting moiety) is present on the surface of the red blood cell-derived vesicle at a density of at least about 30,000 per vesicle.

Most preferably, the at least one targeting ligand (or targeting moiety) is present on the surface of the red blood cell-derived vesicle at a density of at least about 40,000 per vesicle.

Most preferably, the at least one targeting ligand (or targeting moiety) is present on the surface of the red blood cell-derived vesicle at a density of at least about 50,000 per vesicle.

Most preferably, the at least one targeting ligand (or targeting moiety) is present on the surface of the red blood cell-derived vesicle at a density of about 100,000 per vesicle.

Most preferably, the at least one targeting ligand (or targeting moiety) is present on the surface of the red blood cell-derived vesicle at a density of about 1,000,000 per vesicle.

Most preferably, the at least one targeting ligand (or targeting moiety) is present on the surface of the red blood cell-derived vesicle at a density of about 10,000,000 per vesicle.

Most preferably, the at least one targeting ligand (or targeting moiety) is present on the surface of the red blood cell-derived vesicle at a density of about 100 per vesicle.

Advantageously, the inventors have developed novel multifunctional red blood cell-derived vesicles (RBCVs), which enable selective delivery of thrombolytics to a blood clot site and controlled release locally. This targeted delivery can enhance clot disrupting efficacy, limit drug dose and attenuate off-target bleeding side effects. The RBCVs may enable more patients with thrombotic diseases to receive safer and more effective treatment. In addition, the RBCVs act to provide a support surface for anchorage of controllable amounts of the at least one targeting ligand (or targeting moiety). The resulting multi-arm nanovesicles have superior selectivity and strong binding with activated platelets.

The use of the RBCVs disclosed herein, provides for a triggered release mechanism. Before reaching a thrombus, RBCVs can: protect thrombolytic agents in the blood circulation, leading to considerably improved stability and prolonged half-life and temporarily suppress thrombolytic activity, leading to reduced haemorrhagic side effects. Upon selective binding to activated platelets, the RBCVs can fuse with the activated platelet membrane, leading to rapid and efficient release of thrombolytics. This is favourable for treatment of acute events including but not limited to ischemic stroke which requires immediate drug action. It may also be favourable for lysis of both fibrin-rich (responsive to tPA) and platelet-rich blood clots (resistant to tPA), thus with broader potential clinical applications.

In summary, therefore, the novel RBCVs are advantageously and preferably configured to: (i) encapsulate and protect drugs in the bloodstream with considerably improved stability and elongated half-life, (ii) temporarily suppress thrombolytic activity in the bloodstream and thus reduce the risk of systemic bleeding, (iii) target drugs to the occluded vessel and thus improve its efficacy without increasing off-targets, and (iv) selectively bind to activated platelets, which can cause efficient and rapid controlled drug release locally as a result of membrane fusion; and (v) enhance penetration of drugs into clots and thus lead to efficient recanalisation.

The invention also extends to a method of producing the red blood cell-derived vesicle of the first aspect.

Accordingly, in a second aspect of the invention, there is provided a method of preparing a red blood cell-derived vesicle (RBCV) comprising an encapsulated active agent, the method comprising:
(i) contacting a red blood cell with a hypotonic solution to produce a red blood cell ghost; and
(ii) encapsulating an active agent using the red blood cell ghost, to thereby produce a red blood cell-derived vesicle comprising an encapsulated active agent.

The term “red blood cell ghost” can refer to a red blood cell that has had its haemoglobin removed, such that under a microscope, the cell appears pale with no internal content in a blood smear.

Hence, a “red blood cell ghost” can therefore refer to a red blood cell that is substantially free of haemoglobin. For example, a “red blood cell ghost” may refer to a red blood cell that has at least 50% of its haemoglobin removed, at least 60% of haemoglobin removed, at least 70% of haemoglobin removed, at least 80% of haemoglobin removed, or at least 90% of haemoglobin removed. A “red blood cell ghost” may refer to a red blood cell that has at least 95% of haemoglobin removed, at least 96% of haemoglobin removed, at least 97% of haemoglobin removed at least 98% of haemoglobin removed, at least 99% of haemoglobin removed or 100% of haemoglobin removed.

Step (i) may comprise contacting the red blood cells with a solution that is hypotonic when compared to the cytosol of the red blood cell. Preferably, the hypotonic solution acts to rupture the red blood cell membrane. Most preferably, the hypotonic solution comprises ethylene diamine tetraacetic acid (EDTA).

The hypotonic solution may be a hypotonic PBS solution. Preferably, the hypotonic solution may be a PBS solution comprising between about 0.1 x and 0.5 x concentration of PBS. Most preferably, the hypotonic solution may be a PBS solution comprising about 0.25 x concentration of PBS.

Step (i) may further comprise centrifuging the solution and removing the supernatant. Preferably, centrifugation is performed with a centrifugal force of about 1 g to 25000 g, 10 g to 25000 g, 100 g to 25000 g, 1000 g to 25000 g, 10000 to 25000 g, 1 g to 2000 g, 10 g to 2000 g, 100 g to 2000 g, 1000 g to 2000 g, 10000 to 2000 g, 1 g to 15000 g, 10 g to 15000 g, 100 g to 15000 g, 1000 g to 15000 g, 10000 to 15000 g, 1 g to 10000 g, 10 g to 10000 g, 100 g to 10000 g, 1000 g to 10000 g, 1 g to 5000 g, 10 g to 5000 g, 100 g to 5000 g, 1000 g to 5000 g, 1 g to 4000 g, 10 g to 4000 g, 100 g to 4000 g, 1000 g to 4000 g, 1 g to 3000 g, 10 g to 3000 g, 100 g to 3000 g, 1000 g to 3000 g.

Preferably, centrifugation is performed with a centrifugal force of about 1000 g to about 10000 g. Preferably, centrifugation is performed with a centrifugal force of about 2000 g to about 6000 g. Preferably, centrifugation is performed with a centrifugal force of about 3000 g to about 5000 g. Most preferably, centrifugation is performed with a centrifugal force of about 4000 g.

Centrifugation may be performed at a temperature of between 0° C. and 20° C. Preferably, centrifugation is performed at a temperature of between about o °C and 15° C., or between about 0° C. and 10° C., or between about 0° C. and 5° C. Preferably, centrifugation may be performed at a temperature of between about 1° C. and 10° C., or between about 1° C. and 9° C., or between about 1° C. and 8° C., or between about 1° C. and 7° C., or between about 1° C. and 6° C., or between about 1° C. and 5° C., or between about 1° C. and 4° C.

Preferably, centrifugation may be performed at a temperature of between about 2° C. and 10° C., or between about 2° C. and 9° C., or between about 2° C. and 8° C., or between about 2° C. and 7° C., or between about 2° C. and 6° C., or between about 2° C. and 5° C., or between about 2° C. and 4℃. Most preferably, centrifugation is performed at a temperature of about 4° C.

Step (i) may be repeated at least once. Step (i) may be repeated at least 2, 3, 4, 5, 6, 7, 8, 9 or 10 times. Preferably, step (i) is repeated at least two times. Preferably, step (i) is repeated at least three times. Preferably, step (i) is repeated at least four times. Preferably, step (i) is repeated at least five times. Preferably, step (i) is repeated at least six times.

Preferably, step (i) is repeated until the supernatant is substantially free of red colour.

Preferably, the supernatant resulting from centrifugation is removed and the resulting pellet is re-suspended in a buffer solution, preferably a phosphate-buffered saline (PBS) buffer solution.

Preferably, the buffer solution has a pH of between about 3 and 11. Preferably, the buffer solution has a pH of between about 4 and 10. Preferably, the buffer solution has a pH of between about 5 and 9. Preferably, the buffer solution has a pH of between about 6 and 8. Preferably, the buffer solution has a pH of between about 6.5 and 8.5. Preferably, the buffer solution has a pH of between about 7 and 8. Most preferably the buffer solution has a pH of about 7.4.

Step (ii) preferably comprises encapsulating the active agent by the red blood cell ghost, to thereby produce a red blood cell-derived vesicle comprising an encapsulated active agent. Step (ii) may comprise contacting the red blood cell ghost resulting from step (i) with an active agent and, optionally, at least one targeting ligand (or targeting moiety). Preferably, step (ii) may comprise contacting the red blood cell ghost resulting from step (i) with a plurality of targeting ligands (or targeting moieties).

Contacting the red blood cell ghost resulting from step (i) with an active agent and a targeting ligand (or targeting moiety), may occur subsequently, such that the red blood cell ghost resulting from step (i) may be contacted with an active agent and then subsequently contacted with a targeting ligand (or targeting moiety).

In another embodiment, contacting of the red blood cell ghost with the active agent and targeting ligand (or targeting moiety) may occur in the same step, or substantially simultaneously.

Contacting the red blood cell ghost resulting from step (i) with an active agent and a targeting ligand (or targeting moiety), may occur substantially simultaneously.

The targeting ligand (or targeting moiety) may be conjugated to a lipid. For example, 1, ₂-Distearoyl-sn-glycero-₃-phosphoethanolamine (DSPE) or egg phosphatidylcholine (EPC).

The targeting ligand (or targeting moiety) may be provided as a film, which targets lipid, and is formed by removal of the organic solvent and then may be hydrated by contacting the film with the active agent containing red blood cell ghost solution. Alternatively, the film may be formed by removal of the organic solvent and then may be hydrated by contacting the film with, separately, the active agent solution and a red blood cell ghost solution.

The film of targeting ligand (or targeting moiety) that targets lipid-may be formed by dissolving the targeting ligand (or targeting moiety) conjugated to a lipid in a solvent and removing the solvent by evaporation.

The solvent may be chloroform or dichloromethane.

Evaporation may be performed by rotary evaporation.

The targeting ligand (or targeting moiety) may be hydrated by contacting the film with the active agent-containing red blood cell ghost solution, or with, separately, the active agent solution and a red blood cell ghost solution, at a temperature of between about 20° C. to about 60° C. Preferably, the film may be hydrated by contacting the film with the active agent-containing red blood cell ghost solution, or with, separately, the active agent solution and a red blood cell ghost solution, at a temperature of between about 30° C. to about 50° C. More preferably, the film may be hydrated by contacting the film with the active agent-containing red blood cell ghost solution, or with separately, the active agent solution and a red blood cell ghost solution at a temperature of between about 35° C. to about 45° C.

Most preferably, the film may be hydrated by contacting the film with the active agent-containing red blood cell ghost solution, or with, separately, the active agent solution and a red blood cell ghost solution, at a temperature of about 40° C.

Preferably, encapsulating step ii) may be performed by sonicating the red blood cell ghost resulting from step i) in the presence of the active agent and/or the targeting ligand (or targeting moiety).

Sonication may be performed for between 1 and 60 minutes, between 10 and 60 minutes, between 20 and 60 minutes, or between 30 and 60 minutes. Sonication may be performed for between about 1 and 50 minutes, about 10 and 50 minutes, about 20 and 50 minutes, about 30 and 50 minutes. Sonicating may be performed for between about 1 and 40 minutes, between about 10 and 40 minutes, between about 20 and 40 minutes, or between about 30 and 40 minutes. Preferably, sonication is performed for between about 20 and 40 minutes. More preferably, sonication is performed for between about 25 and 35 minutes. Most preferably, sonication is performed for about 30 minutes.

Step (ii) may further comprise extrusion through a filter at least once.

The extrusion step may be performed at least 1, 2, 3, 4, 5, 10, 15, 20 or 25 times. Preferably, the extrusion step is performed at least once. Preferably, extrusion step is performed at least 3 times. Preferably, extrusion step is performed at least 5 times. Preferably, extrusion step is performed at least 10 times. Preferably, extrusion step is performed at least 15 times. Preferably, extrusion step is performed at least 20 times. Preferably, extrusion step is performed 21 times.

The filter may comprise a pore size of between about 1 nm and 1000 nm, between about 10 nm and 1000 nm, or between about 100 nm and 1000 nm. Preferably, the filter may comprise a pore size of between about 100 nm and 1000 nm. Preferably, the filter may comprise a pore size of between about 100 nm and 500 nm. Preferably, the filter may comprise a pore size of between 100 nm and 400 nm. Preferably, the filter may comprise a pore size of between about 100 nm and 300 nm. Preferably, the filter may comprise a pore size of between about 100 nm and 200 nm. Preferably, the filter may comprise a pore size of between about 150 nm and 350 nm. Preferably, the filter may comprise a pore size of between about 150 nm and 300 nm. Preferably, the filter may comprise a pore size of between about 150 nm and 250 nm. More preferably, the filter may comprise a pore size of about 200 nm.

Preferably, the filter is a polycarbonate membrane.

Step (ii) may further comprise ultracentrifugation to remove the un-encapsulated active agent.

Preferably, centrifugation is performed with a centrifugal force of about 1 g to 25000 g, 10 g to 25000 g, 100 g to 25000 g, 1000 g to 25000 g, 10000 to 25000 g, 1 g to 20000 g, 10 g to 20000 g, 100 g to 20000 g, 1000 g to 20000 g, 10000 to 20000 g, 1 g to 15000 g, 10 g to 15000 g, 100 g to 15000 g, 1000 g to 15000 g, 10000 g to 15000 g, 1 g to 10000 g, 10 g to 10000 g, 100 g to 10000 g, 1000 g to 10000 g, 1 g to 5000 g, 10 g to 5000 g, 100 g to 5000 g, 1000 g to 5000 g, 1 g to 4000 g, 10 g to 4000 g, 100 g to 4000 g, 1000 g to 4000 g, 1 g to 3000 g, 10 g to 3000 g, 100 g to 3000 g, 1000 g to 3000 g.

Preferably, centrifugation is performed with a centrifugal force of about 1000 g to about 10000 g. Preferably, centrifugation is performed with a centrifugal force of about 2000 g to about 6000 g. Preferably, centrifugation is performed with a centrifugal force of about 3000 g to about 5000 g. Most preferably, centrifugation is performed with a centrifugal force of about 4000 g.

Preferably, ultracentrifugation may be performed at a temperature of between about 0° C. and 15° C., or between about 0° C. and 10° C., or between about 0° C. and 5° C. Preferably, centrifugation may be performed at a temperature of between about 1° C. s and 10° C., or between about 1° C. and 9° C., or between about 1° C. and 8° C., or between about 1° C. and 7° C., or between about 1° C. and 6° C., or between about 1° C. and 5° C., or between about 1° C. and 4° C.

Preferably, ultracentrifugation may be performed at a temperature of between about 2° C. and 10° C., or between about 2° C. and 9° C., or between about 2° C. and 8° C., or between about 2° C. and 7° C., or between about 2° C. and 6° C., or between about 2° C. and 5° C., or between about 2° C. and 4° C.

Preferably, ultracentrifugation is performed at about 4° C.

Hence, the method of the second aspect may comprise:
(i) a) contacting a red blood cell with a hypotonic solution;
b) centrifuging the solution resulting from step a) and removing the supernatant; and
c) re-suspending the pellet resulting from step b) in a buffer solution, thereby producing a red blood cell ghost; and
(ii) encapsulating an active agent using the red blood cell ghost by;
   a) contacting the red blood cell ghost resulting from step i) with an active agent;
   b) sonicating the mixture of step a); and
   c) extruding the mixture resulting from step b) through a filter at least once; and
   d) ultracentrifugating the filtrate resulting from step c) to remove the un-encapsulated active agent,
thereby producing a red blood cell-derived vesicle comprising an encapsulated active agent.

The method of the second aspect may comprise:
(i) a) contacting a red blood cell with a hypotonic solution;
b) centrifuging the solution resulting from step a) and removing the supernatant; and
c) re-suspending the pellet resulting from step b) in a buffer solution thereby producing a red blood cell ghost; and
(ii) encapsulating an active agent using the red blood cell ghost by;
   a) contacting the red blood cell ghost resulting from step i) with an active agent and a targeting ligand (or targeting moiety);
   b) sonicating the mixture resulting from step a);
   c) extruding the mixture resulting from step b) through a filter at least once; and
   d) ultracentrifugating the filtrate resulting from step c) to remove the un-encapsulated active agent,
thereby producing a red blood cell-derived vesicle comprising an encapsulated active agent, with the at least one targeting ligand (or targeting moiety) attached to the surface thereto.

The method of the second aspect may comprise:
(i) a) contacting a red blood cell with a hypotonic solution;
b) centrifuging the solution resulting from step a) and removing the supernatant; and
c) re-suspending the pellet resulting from step b) in a buffer solution thereby producing a red blood cell ghost; and
(ii) encapsulating an active agent using the red blood cell ghost by;
   a) contacting the red blood cell ghost resulting from step i) with an active agent;
   b) contacting the mixture resulting from step a) with a targeting ligand (or targeting moiety);
   c) sonicating the mixture resulting from step b);
   d) extruding the mixture resulting from step c) through a filter at least once; and
   e) ultracentrifugating the filtrate resulting from step d) to remove un-encapsulated active agent,
thereby producing a red blood cell-derived vesicle comprising an active agent, with at least one targeting ligand (or targeting moiety) attached to the surface thereto.

Preferably, the active agent and the cell targeting ligand are as defined in the first aspect.

In a third aspect, there is provided a red blood cell-derived vesicle obtained, or obtainable, by the method of the second aspect.

In a fourth aspect of the invention, there is provided a pharmaceutical composition comprising the red blood cell-derived vesicle according to the first or third aspect, and a pharmaceutically acceptable excipient.

In a fifth aspect of the invention, there is provided a method of preparing the pharmaceutical composition according to the fourth aspect, the method comprising contacting the red blood cell-derived vesicle according to the first or third aspect, with a pharmaceutically acceptable excipient.

Advantageously, the red blood cell-derived vesicle of the invention provides for an improved means of delivering therapeutic agents in a targeted manner.

Thus, in a sixth aspect of the invention, there is provided a red blood cell-derived vesicle according to the first or third aspect, or the pharmaceutical composition according to the fourth aspect, for use in therapy.

In a seventh aspect of the invention, there is provided a red blood cell-derived vesicle according to the first or third aspect, or the pharmaceutical composition according to the fourth aspect, for use in thrombolytic therapy, optionally for use in treating, preventing, or reducing a blood clot.

In an eighth aspect, there is provided a method of treating, preventing, or reducing a blood clot, the method comprising administering, or having administered, to a subject in need thereof, a therapeutic amount of the red blood cell vesicle of the first or third aspect, or the pharmaceutical composition of the fourth aspect.

In a ninth aspect, there is provided a method of treating, preventing or ameliorating a thrombotic disorder, the method comprising administering, or having administered, to a subject in need thereof, a therapeutic amount of the red blood cell vesicle of the first or third aspect or the pharmaceutical composition of the fourth aspect.

The thrombolytic therapy may relate to treatment of a thrombotic disorder selected from the group consisting of: ischemic stroke; myocardial infarction and pulmonary embolism.

In a tenth aspect, there is provided an anti-clotting agent comprising a red blood cell-derived vesicle according to the first or third aspect.

Preferably, the anti-clotting agent may be an anti-platelet agent.

The red blood cell-derived vesicle or the pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch or any other suitable form that may be administered to a person or animal in need of treatment. It will be appreciated that the vehicle of medicaments according to the invention should be one which is well-tolerated by the subject to whom it is given.

The red blood cell-derived vesicle or the pharmaceutical composition of the invention may also be incorporated within a slow- or delayed-release device. Such devices may, for example, be inserted on or under the skin, and the medicament may be released over weeks or even months. The device may be located at least adjacent the treatment site.

In a preferred embodiment, however, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion).

It will be appreciated that the amount of red blood cell-derived vesicle or the pharmaceutical composition that is required is determined by its biological activity and bioavailability, which in turn depends on the mode of administration, the physiochemical properties of the red blood cell-derived vesicle or the pharmaceutical composition and whether it is being used as a monotherapy or in a combined therapy.

The frequency of administration will also be influenced by the half-life of the active agent within the subject being treated. Optimal dosages to be administered may be determined by those skilled in the art, and will vary with the red blood cell-derived vesicle or the pharmaceutical composition in use, the strength of the pharmaceutical composition, the mode of administration, and the type of treatment. Additional factors depending on the particular subject being treated will result in a need to adjust dosages, including subject age, weight, gender, diet, and time of administration.

Generally, a dose of between 0.001 µg/kg of body weight and 10 mg/kg of body weight, or between 0.01 µg/kg of body weight and 1 mg/kg of body weight, of the red blood cell-derived vesicle or the pharmaceutical composition, or the active agent may be used.

Doses may be given as a single administration (e.g. a single injection). Alternatively, the red blood cell-derived vesicle or the pharmaceutical composition may require more than one administration. As an example, the red blood cell-derived vesicle or the pharmaceutical composition, or the active agent, may be administered as two (or more depending upon red blood cell-derived vesicle, or the active agent) doses of 0.07 µg and 700 mg kg (i.e. assuming a body weight of 70 kg). Alternatively, a slow release device may be used to provide optimal doses of the red blood cell-derived vesicle or the pharmaceutical composition, or the active agent, according to the invention to a patient without the need to administer repeated. Routes of administration may incorporate intravenous, intradermal subcutaneous, or intramuscular routes of injection.

Known procedures, such as those conventionally employed by the pharmaceutical industry (e.g. in vivo experimentation, clinical trials, etc.), may be used to form specific formulations of the red blood cell-derived vesicle according to the invention and precise therapeutic regimes (such as doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of red blood cell-derived vesicle or the pharmaceutical composition is any amount which, when administered to a subject, is the amount of the aforementioned that is needed to produce a therapeutic effect.

For example, the red blood cell-derived vesicle and the pharmaceutical composition, or the active agent, of the invention may be used from about 0.01 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the agent (e.g. the red blood cell-derived vesicle of the invention) may be mixed with a vehicle having the necessary compression properties in suitable proportions and compacted in the shape and size desired. The pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurised compositions. The red blood cell-derived vesicle according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilisers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurised compositions can be a halogenated hydrocarbon or other pharmaceutically acceptable propellant.

Liquid pharmaceutical compositions, which are sterile solutions or suspensions, can be utilised by, for example, intramuscular, intrathecal, epidural, intraperitoneal, intravenous and particularly subcutaneous injection. The red blood cell-derived vesicle of the invention may be prepared as any appropriate sterile injectable medium.

The red blood cell-derived vesicle and the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerised with ethylene oxide) and the like. The red blood cell-derived vesicle of the invention and the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-16 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22,4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty = 15.0, Gap Extension Penalty = 6.66, and Matrix = Identity. For protein alignments: Gap Open Penalty = 10.0, Gap Extension Penalty = 0.2, and Matrix = Gonnet. For DNA and Protein alignments: ENDGAP = -1, and GAPDIST = 4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:- Sequence Identity = (N/T)*100.

Alternative methods for identifying similar sequences will be known to those skilled in the art. For example, a substantially similar nucleotide sequence will be encoded by a sequence which hybridises to DNA sequences or their complements under stringent conditions. By stringent conditions, the inventors mean the nucleotide hybridises to filter-bound DNA or RNA in 3 x sodium chloride/sodium citrate (SSC) at approximately 45° C. followed by at least one wash in 0.2 x SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos:1 to 16.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:-

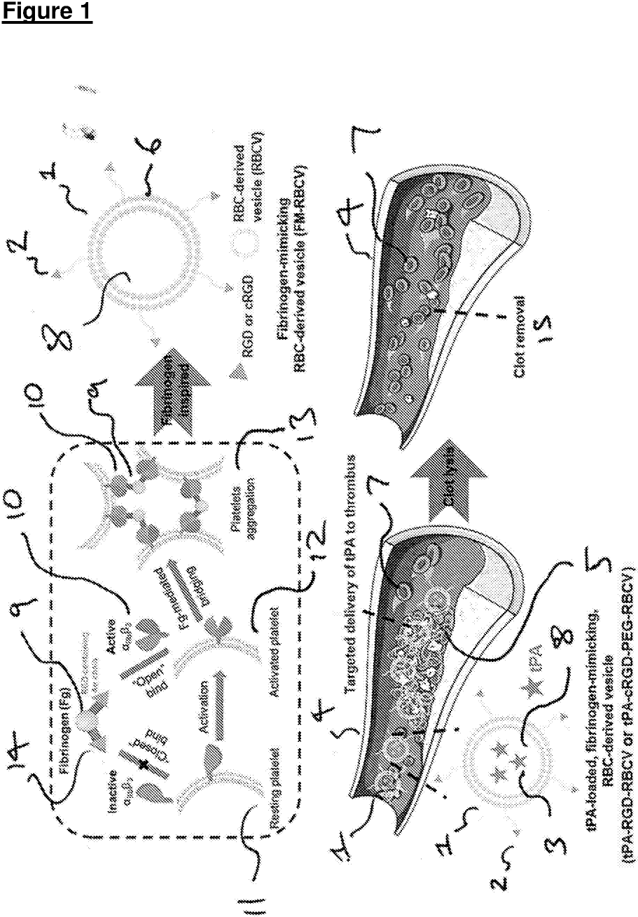

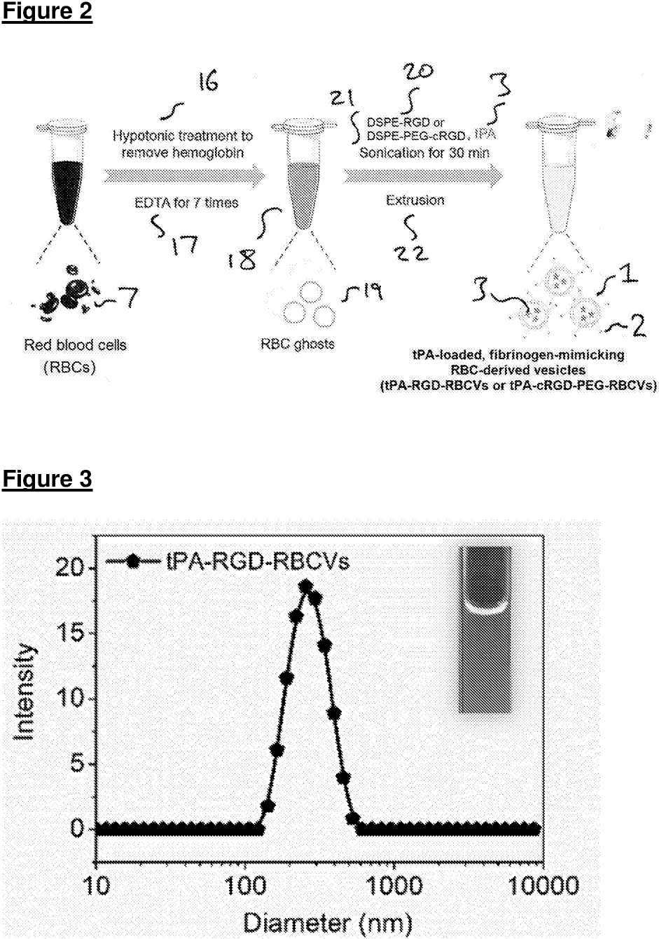

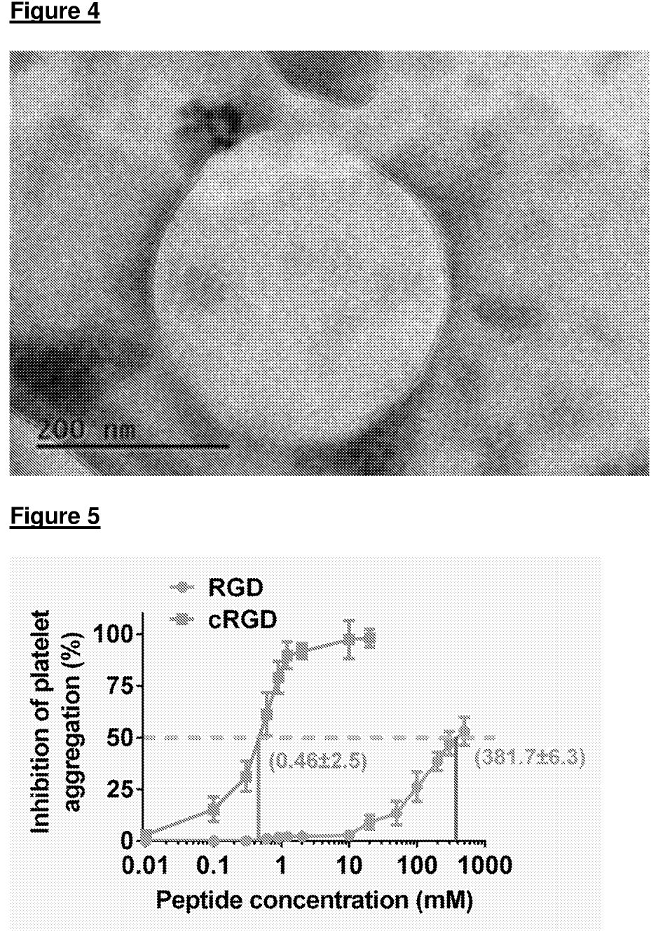

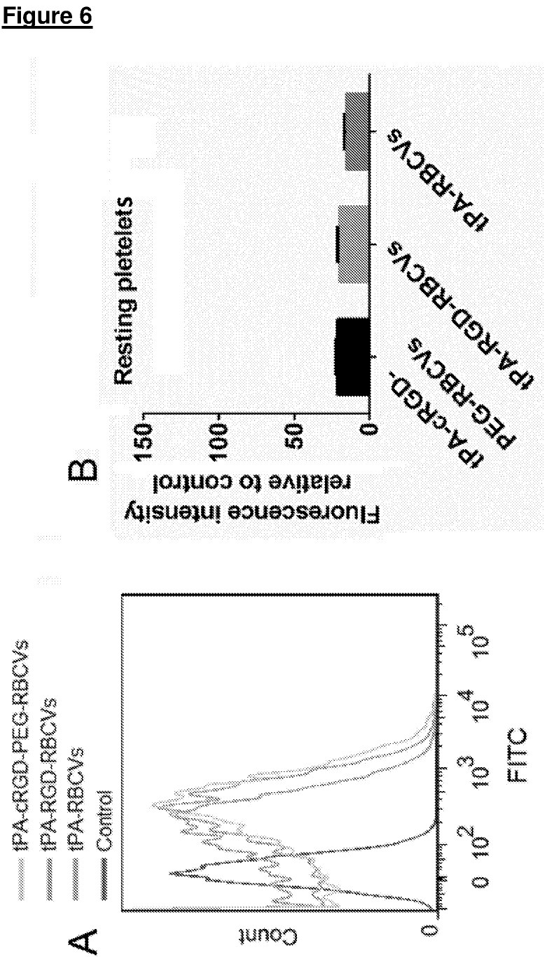

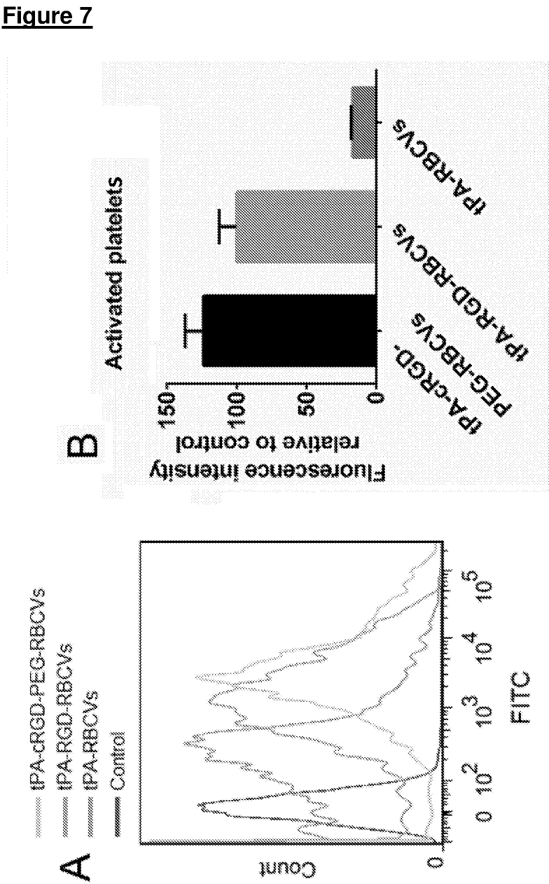

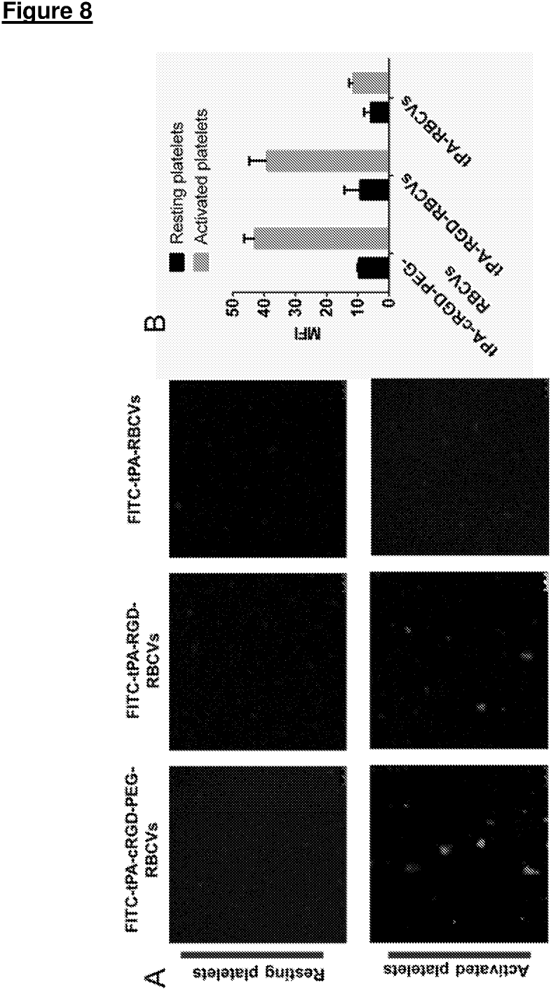

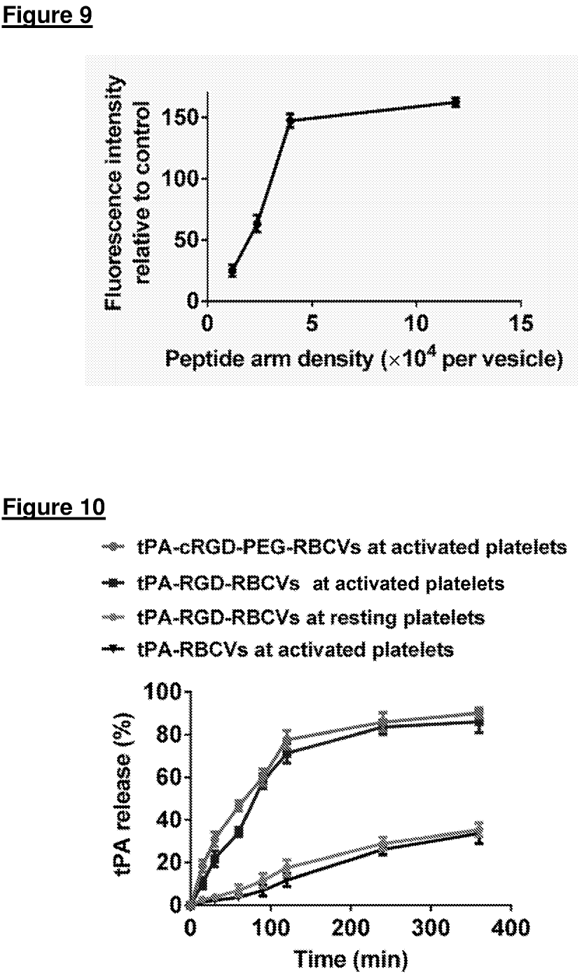

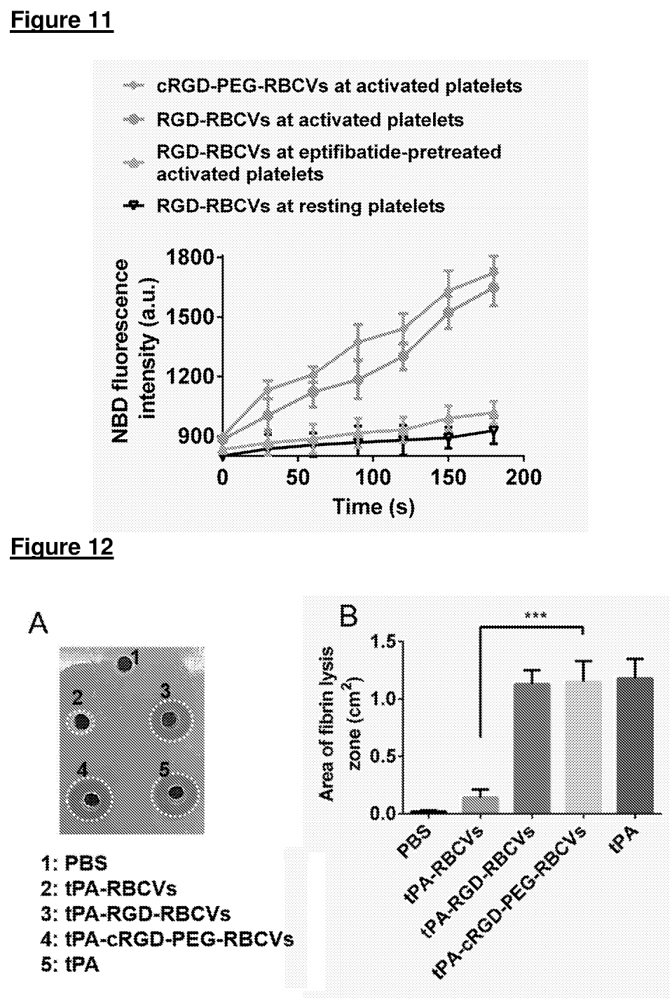

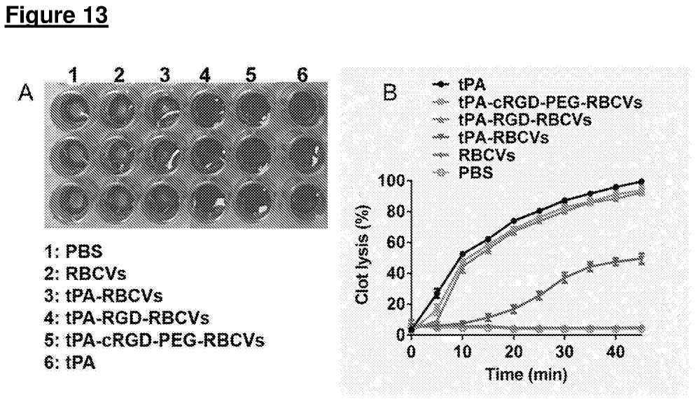

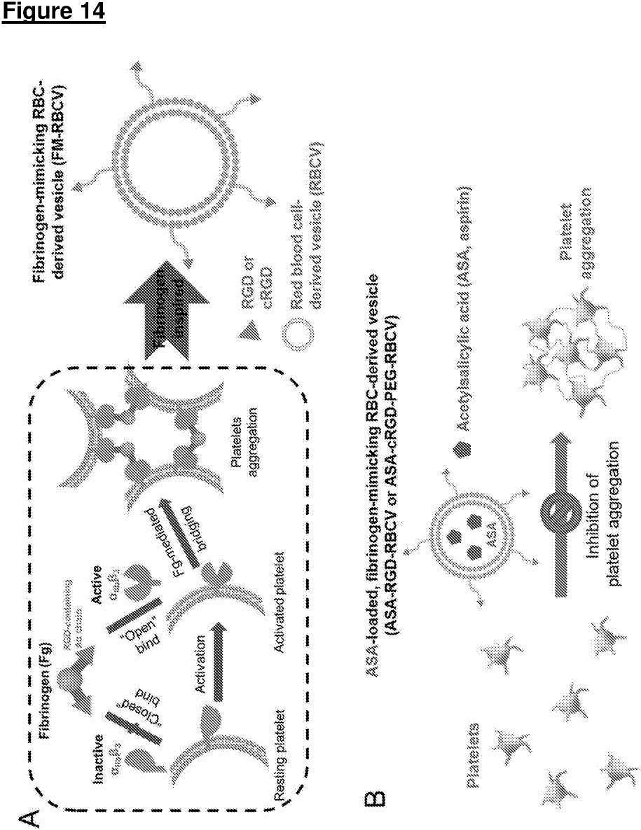

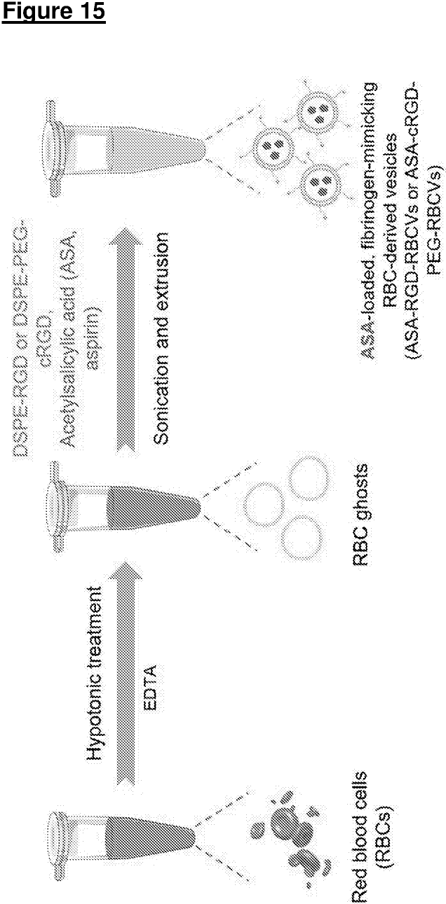

FIG. 1 shows a schematic illustration of the mechanism of platelet aggregation (top left), an embodiment of a fibrinogen-mimicking red blood cell vesicle (RBCV) according to the invention (FM-RBCV, top right), and also targeted delivery of tissue plasminogen activator (tPA) to a blood clot using the tPA-loaded, fibrinogen-mimicking RBCV (tPA-RGD-RBCV or tPA-cRGD-PEG-RBCV) for targeted thrombolysis to remove the blood clot (bottom left and right).

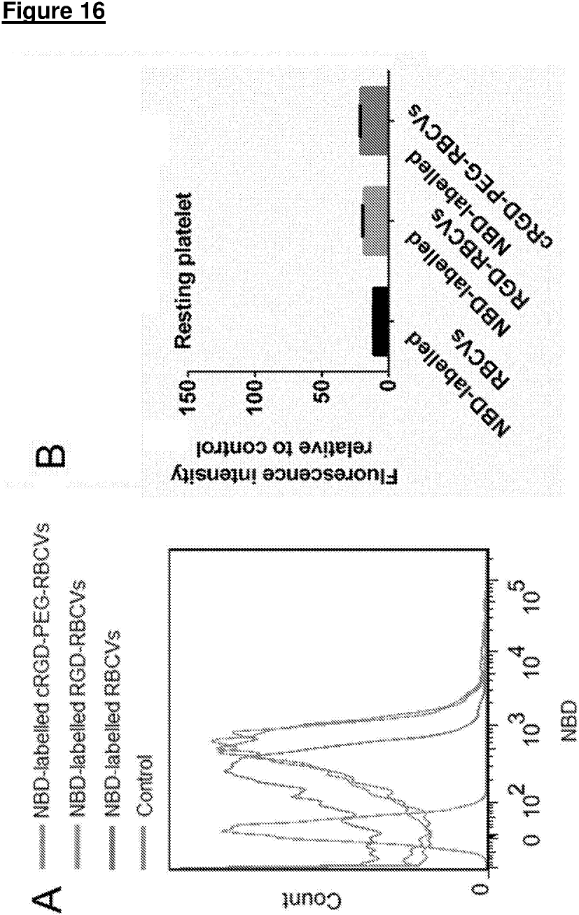

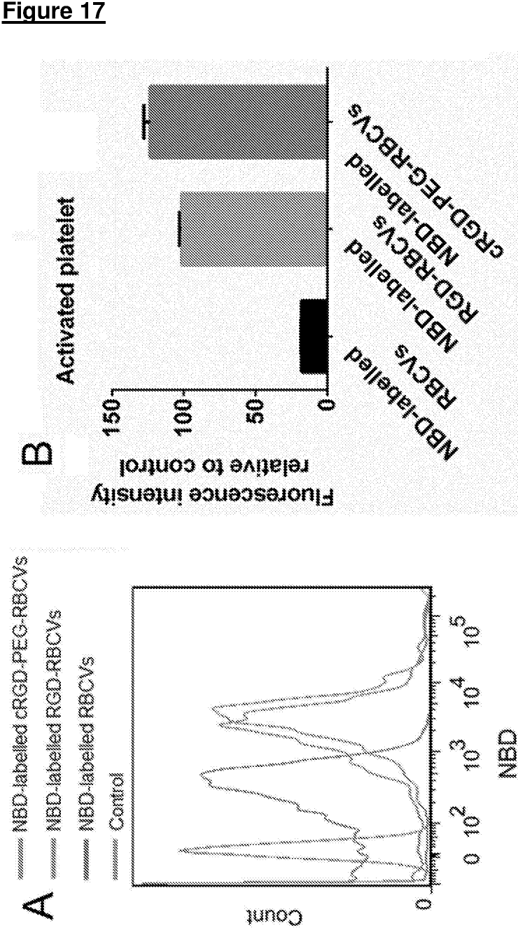

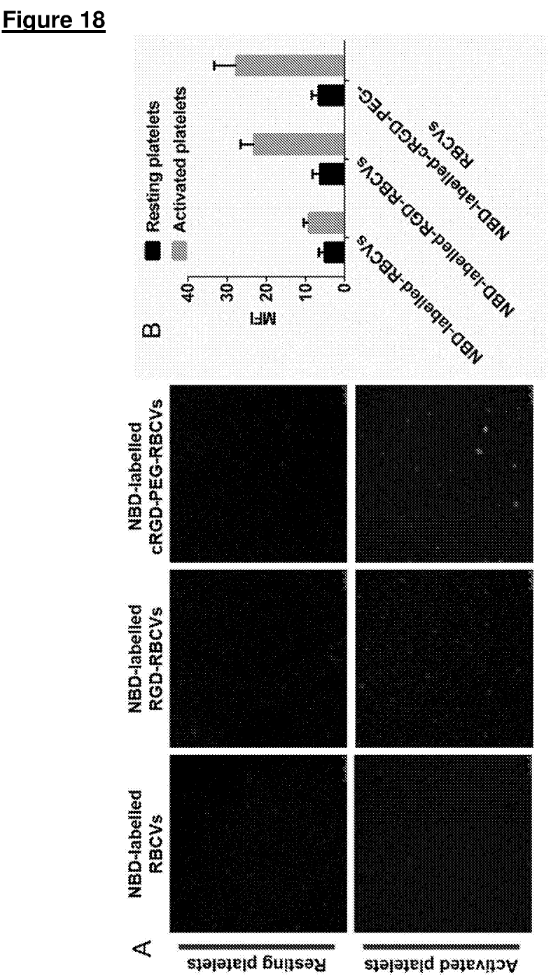

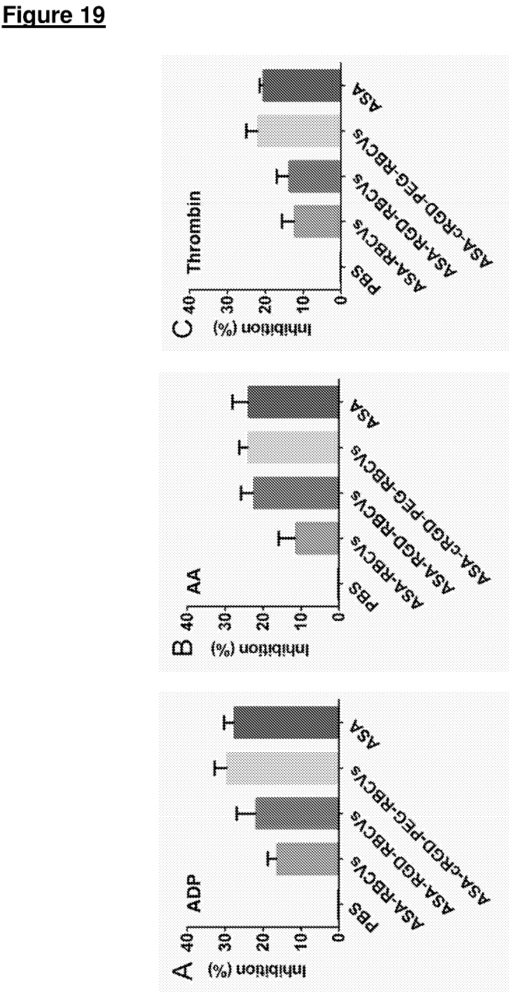

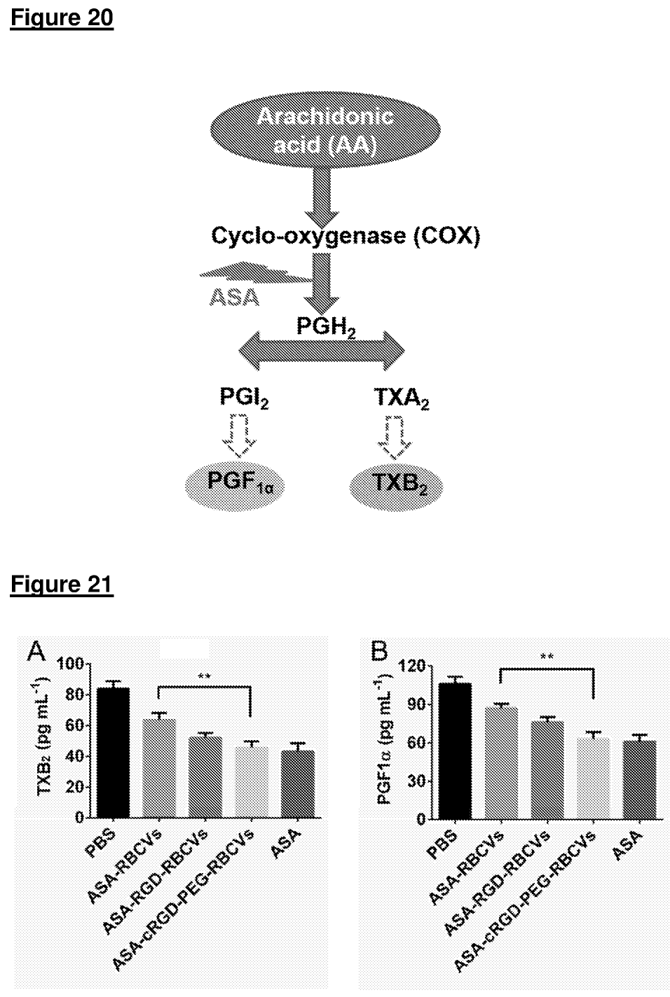

FIG. 2 shows a schematic illustration of one embodiment of the preparation of tPA-loaded, fibrinogen-mimicking, RBC-derived vesicles (tPA-RGD-RBCVs or tPA-cRGD-PEG-RBCVs) according to the invention.

FIG. 3 shows a typical dynamic light scattering (DLS) plot of tPA-RGD-RBCVs in pH 7.4 PBS buffer. Inset: representative photograph of tPA-RGD-RBCVs solution.

FIG. 4 shows a representative transmission electron microscope (TEM) image of the tPA-RGD-RBCVs according to the invention. The scale bar was 200 nm.

FIG. 5 shows the half maximal inhibitory concentration (IC50) of linear RGD and cyclic RGD (cRGD) measured by the platelet aggregation assay.

FIG. 6 shows (A) Typical flow cytometry histogram profiles of resting platelets incubated with FITC-labelled tPA-RBCVs, FITC-labelled tPA-RGD-RBCVs and FITC-labelled tPA-cRGD-PEG-RBCVs, respectively. Resting platelets treated with pH 7.4 PBS buffer were used as the control. (B) Fluorescence intensities of resting platelets treated with FITC-labelled tPA-RBCVs, FITC-labelled tPA-RGD-RBCVs and FITC-labelled tPA-cRGD-PEG-RBCVs relative to the control, respectively.

FIG. 7 shows (A) Typical flow cytometry histogram profiles of activated platelets incubated with FITC-labelled tPA-RBCVs, FITC-labelled tPA-RGD-RBCVs and FITC-labelled tPA-cRGD-PEG-RBCVs, respectively. The platelets treated with pH 7.4 PBS buffer were used as the control. (B) Fluorescence intensities of activated platelets respectively treated with FITC-labelled tPA-RBCVs, FITC-labelled tPA-RGD-RBCVs and FITC-labelled tPA-cRGD-PEG-RBCVs relative to the control.

FIG. 8 shows (A) Typical confocal laser scanning microscopy images of resting or activated platelets incubated with FITC-labelled tPA-RBCVs, FITC-labelled tPA-RGD-RBCVs and FITC-labelled tPA-cRGD-PEG-RBCVs, respectively. (B) Mean fluorescence intensity (MFI) in the fluorescence images of resting or activated platelets after incubation with FITC-labelled tPA-RBCVs, FITC-labelled tPA-RGD-RBCVs and FITC-labelled tPA-cRGD-PEG-RBCVs, respectively.

FIG. 9 shows relative fluorescence intensity of activated platelets incubated with the FITC-labelled tPA-RGD-RBCVs containing different RGD peptide arm densities as measured by flow cytometry.

FIG. 10 shows tPA release profiles of tPA-RBCVs, tPA-RGD-RBCVs and tPA-cRGD-PEG-RBCVs in the presence of resting or activated platelets.

FIG. 11 shows NBD fluorescence intensity after incubation of RGD-RBCVs or cRGD-PEG-RBCVs (containing 0.01 mmol NBD-PE and 0.01 mmol Rhod-PE) with resting platelets, activated platelets or eptifibatide-pretreated activated platelets, respectively, at various time intervals.

FIG. 12 shows (A) Representative photograph of fibrin clots treated with PBS buffer only, tPA-RBCVs, tPA-RGD-RBCVs, tPA-cRGD-PEG-RBCVs and free tPA, respectively, in the presence of activated platelets. (B) The area of the fibrin lysis ring after treatment with PBS buffer only, tPA-RBCVs, tPA-RGD-RBCVs, tPA-cRGD-PEG-RBCVs or free tPA in the presence of activated platelets. Statistical analysis was performed using the Student's t-test. The triple asterisk symbol (***) denotes p < 0.001.

FIG. 13 shows (A) Representative photograph of Halo blood clots after treatment with PBS buffer only, RBCVs, tPA-RBCVs, tPA-RGD-RBCVs, tPA-cRGD-PEG-RBCVs and free tPA, respectively. (B) Time-dependent clot lysis in the Halo model after treatment with PBS buffer only, RBCVs, tPA-RBCVs, tPA-RGD-RBCVs, tPA-cRGD-PEG-RBCVs and free tPA, respectively.

FIG. 14 shows (A) A schematic illustration of the mechanism of platelet aggregation (left) and an embodiment of a fibrinogen-mimicking RBCV according to the invention (right). (B) A schematic illustration of the acetylsalicylic acid (ASA, Aspirin)-loaded, fibrinogen-mimicking RBCV (ASA-RGD-RBCV or ASA-cRGD-PEG-RBCV) for inhibition of platelet aggregation.

FIG. 15 shows a schematic illustration of one embodiment of the preparation of ASA-loaded, fibrinogen-mimicking RBCVs (ASA-RGD-RBCVs or ASA-cRGD-PEG-RBCVs) according to the invention.

FIG. 16 shows (A) Typical flow cytometry histogram profiles of resting platelets incubated with NBD-labelled RBCVs, NBD-labelled RGD-RBCVs and NBD-labelled cRGD-PEG-RBCVs, respectively. The platelets treated with pH 7.4 PBS buffer were used as the control. (B) Fluorescence intensities of activated platelets treated with NBD-labelled RBCVs, NBD-labelled RGD-RBCVs, and NBD-labelled cRGD-PEG-RBCVs relative to the control, respectively.

FIG. 17 shows (A) Typical flow cytometry histogram profiles of activated platelets incubated with NBD-labelled RBCVs, NBD-labelled RGD-RBCVs and NBD-labelled cRGD-PEG-RBCVs, respectively. The platelets treated with pH 7.4 PBS buffer were used as the control. (B) Fluorescence intensities of activated platelets treated with NBD-labelled RBCVs, NBD-labelled RGD-RBCVs, and NBD-labelled cRGD-PEG-RBCVs relative to the control, respectively.

FIG. 18 shows (A) Typical confocal laser scanning microscopy images of resting or activated platelets incubated with NBD-labelled RBCVs, NBD-labelled RGD-RBCVs and NBD-labelled cRGD-PEG-RBCVs, respectively. (B) Mean fluorescence intensity (MFI) in the fluorescence images of resting or activated platelets after incubation with NBD-labelled RBCVs, NBD-labelled RGD-RBCVs and NBD-labelled cRGD-PEG-RBCVs, respectively.

FIG. 19 shows inhibition ratios of PBS, ASA-RBCVs, ASA-RGD-RBCVs, ASA-cRGD-PEG-RBCVs and ASA against (A) adenosine diphosphate (ADP)-induced, (B) arachidonic acid (AA)-induced or (C) thrombin-induced platelet aggregation.

FIG. 20 shows a schematic illustration of the mechanisms of inhibition of cyclooxygenase (COX) by ASA.

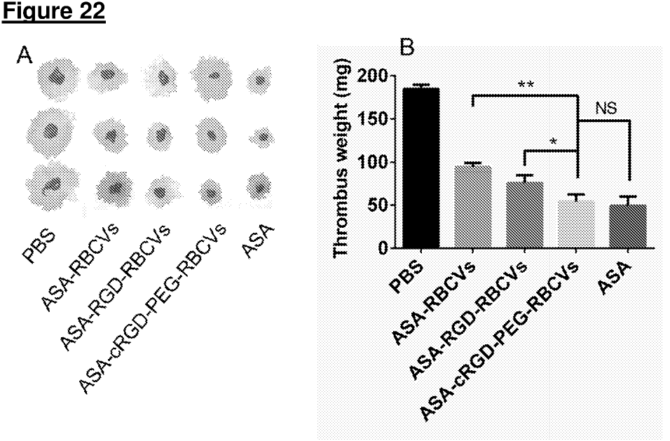

FIG. 21 shows the level of (A) thromboxane B2 (TXB2) and (B) prostaglandin F1α (PGF1α) in plasma after incubation with PBS, ASA-RBCVs, ASA-RGD-RBCVs, ASA-cRGD-PEG-RBCVs and ASA, respectively. Statistical analysis was performed using the Student's t-test. The double asterisk symbol (**) denotes p < 0.01.

FIG. 22 shows (A) a representative photograph of the wet thrombi after treatment with PBS, ASA-RBCVs, ASA-RGD-RBCVs, ASA-cRGD-PEG-RBCVs and ASA, respectively, and (B) the weight of thrombi formed in the presence of PBS, ASA-RBCVs, ASA-RGD-RBCVs, ASA-cRGD-PEG-RBCVs and ASA, respectively. Statistical analysis was performed using the Student's t-test. The single asterisk symbol (*) denotes p < 0.05, the double asterisk symbol (**) denotes p < 0.01, and NS represents no significant difference between two groups.

### EXAMPLES

### Materials and Methods

### Materials

Tissue plasminogen activator (tPA, alteplase) was a product of Boehringer Ingelheim (Germany). 1, 2-distearoyl-sn-glycero-3-phosphoethanolamine-N-[4-(p-(cysarginylglycylaspartate-maleimidomethyl)cyclohexane-carboxamide] (DSPE-RGD), acetylsalicylic acid (aspirin, ASA), 1, 2-distearoyl-sn-glycero-3-phosphoethanolamine-N-[amino(polyethylene glycol)-2000] (DSPE-PEG-NH2), cyclo(Arg-Gly-Asp-d-Phe-Val) (cRGD), 4-dimethylaminopyridine (DMAP), N-hydroxy succinimide (NHS), 1-(3-dimethylaminopropyl)-3-ethyl carbodiimide hydrochloride (EDC), L-α-phosphatidylethanolamine-N-(7-nitro-2-1,3-benzoxadiazol-4-yl) (NBD-PE), L-α-phosphatidylethanolamine-N-(lissamine rhodamine B sulfonyl) (Rhod-PE), Dulbecco's phosphate-buffered saline (D-PBS), sodium chloride (NaCl), calcium chloride (CaCl₂), fluorescein isothiocyanate (FITC), Triton X-100, thrombin, arachidonic acid (AA), adenosine diphosphate (ADP), plasminogen, fibrinogen (Fg), tris(hydroxymethyl) aminomethane, agar, tPA chromogenic activity assay kit S-2251, acid citrate dextrose (ACD), and ethylene diamine tetraacetic acid (EDTA) were purchased from Sigma-Aldrich (Dorset, UK). Centrifugal concentrators were purchased from Fisher Scientific (Loughborough, UK). Sheep blood was obtained from TCS Biosciences Ltd (Buckingham, UK). The extruder set, polycarbonate membranes and filter supports were purchased from Avanti Polar Lipids Inc. (Alabaster, USA). Thromboxane B2 (TXB2) ELISA kit and 6-keto-PGF1α ELISA kit were purchased from Abcam (Cambridge, UK). Chloroform (CHCl₃), hydrochloric acid, sodium hydroxide and other chemicals were obtained from VWR (Lutterworth, UK).

### Preparation and Characterisation of tPA-Loaded, Fibrinogen-Mimicking, Red Blood Cell-Derived Vesicles (tPA-RGD-RBCVs and tPA-cRGD-PEG-RBCVs)

### a) Preparation of RBC-Derived Vesicles (RBCVs)

Briefly, the blood was centrifuged at 3000 rpm for 5 min and then washed with phosphate buffered saline (PBS, pH=7.4) for three times to remove serum. RBCs were re-suspended in 0.2 mM EDTA solution to induce membrane rupture. Subsequently, the cell solution was adjusted to 1×PBS by using 10 × PBS and then centrifuged at 14800 rpm for 7 min at 4° C. to remove the supernatant. The EDTA treatment step was repeated for times until the supernatant was free of red colour. The resulting RBC ghosts were re-suspended in pH 7.4 PBS and then sonicated until a clear and transparent solution of RBCVs was obtained.

In an alternative method to generate red blood cell ghost from red blood cells, an ice bath hypotonic treatment protocol with a few modifications (Hu et al., Proc Natl Acad Sci USA, 2011, 108, 10980-5) was performed. RBCs were washed three times with ice-cold 1 x PBS and centrifuged for 5 min at 3000 rpm at 4° C. The resulting RBC pellet was treated with 0.25 x PBS and incubated in a shaking ice bath for 20 minutes at 50 rpm. The resulting solution was centrifuged at 12,100 rpm at 4° C. for 8 minutes and the supernatant was removed. The resulting pellet of red blood cell ghost was washed once with 1x PBS and resuspended in the same solution before being stored in the fridge.

### b) Preparation of tPA-loaded, Fibrinogen-Mimicking RBCVs (tPA-RGD-RBCVs and tPA-cRGD-PEG-RBCVs)

RBC ghosts were re-suspended in pH 7.4 PBS buffer, which was then added with tPA at a final concentration of 5 mg mL⁻¹. The DSPE-PEG-cRGD lipid was first synthesised by an amidation reaction between the free -NH₂ in DSPE-PEG-NH₂ and the free -COOH in cyclo(Arg-Gly-Asp-d-Phe-Val) (cRGD) peptide. DSPE-RGD lipid or DSPE-PEG-cRGD lipid (5.0 × 10⁻⁷ mol) was dissolved in chloroform in a 25-mL round-bottom flask. A lipid film was formed by removal of the organic solvent with rotary evaporation, and then hydrated with 5 mL of tPA-containing RBC ghost solution at 40° C. for 1 h. The mixture solution was sonicated at 4° C. for 30 min, followed by extrusion for times through the 200-nm polycarbonate membrane by using the extruder set. The unencapsulated tPA was removed by ultracentrifugation (Eppendorf, UK) at 4° C. Meanwhile, tPA-loaded RBCVs without RGD coating (tPA-RBCVs) were synthesised by using the same protocol for comparison.

### c) Vesicle Characterisation

The hydrodynamic size and polydispersity index (PDI) of tPA-cRGD-PEG-RBCVs, tPA-RGD-RBCVs and tPA-RBCVs were measured by dynamic light scattering (DLS, Zetasizer Nano S, Malvern, UK). Their morphology was measured by transmission electron microscopy (TEM, JEOL JEM-2100F, Japan). Their zeta potential was measured by zeta potential analyser (ZetaPALS, Brookhaven, USA).

The nanoparticle tracking analysis (NTA) of 1 mL of RBCVs in pH 7.4 PBS buffer was performed by a NanoSight NS300 equipped with a 532 nm green laser (Malvern, UK).

The tPA content in the vesicles was determined by the chromogenic substrate S-2251 assay. The encapsulation efficiency (EE) was calculated according to the following equation:

$$\text{EE} \left(\%\right) = \frac{{m}_{l}}{{m}_{t}} \times 100$$

where mₗ is the weight of tPA loaded and mₜ is the total weight of tPA in the initial loading solution.

### Binding Affinity with Activated Platelets

### a) Isolation of Platelets

Platelets were isolated from whole blood with anti-coagulant ACD by differential centrifugation. Briefly, the blood was placed into a 2-mL tube and centrifuged at 200 × g for 15 min. Platelet-rich plasma (PRP) was obtained by removing RBCs, and platelets were then collected by centrifugation of PRP at 800 × g for 15 min. Platelets were resuspended in PBS buffer and counted for experimental use.

### b) Preparation of FITC-Labelled tPA

To evaluate the potential of the fibrinogen-mimicking vesicles for targeted tPA delivery and selective thrombolysis, their specific binding to activated platelets was determined by flow cytometry and confocal laser scanning microscopy (CLSM). For the purpose, tPA was fluorescently labelled by fluorescein isothiocyanate (FITC). Briefly, 1 mL of FITC solution in DMSO (1.0 mg mL⁻¹) was added dropwise into 2 mL of tPA solution in PBS buffer at pH 8.0 (1.0 mg mL⁻¹) and stirred at 4° C. in the dark overnight. The mixture was dialysed against pH 7.4 PBS solution for 24 h (MWCO = 3500 Da) and the purified product was stored at 4° C. before use.

### c) Binding Affinity of Free Peptides

Binding affinity of free liner RGD or cRGD peptides to activated platelets can be indicated by a half maximal inhibitory concentration (IC₅₀) value, which is the peptide concentration required to inhibit fibrinogen mediated platelet aggregation in platelet-rich plasma (PRP) by 50%. PRP was incubated with various concentrations of liner RGD or cRGD peptides in the presence of platelet agonist, thrombin (4 µM) for 30 min under stirring. The platelet aggregation percentage was determined with a GloMax-Multi Microplate Multimode Reader (Promega, USA) by measuring the absorbance at 595 nm. The platelet aggregation percentage was calculated by the following equation:

$$\text{Aggregation} \left(\%\right) = \frac{{A}_{0} - {A}_{t}}{{A}_{0}} \times 100$$

where Aₒ is the initial absorbance at 595 nm, and Aₜ is the absorbance at 595 nm after incubation.

The percentage platelet aggregation inhibition was calculated by the following equation:

$$\text{Inhibition} \left(\%\right) = \frac{P {A}_{P B S} - P {A}_{S}}{P {A}_{P B S}} \times 100$$

where PA_{PBS} is the platelet aggregation in the PRP control in the presence of thrombin and PBS only (no peptides), and PAₛ is the platelet aggregation in the PRP sample in the presence of thrombin and peptides.

### d) Determination of Binding Affinity with Activated Platelets by Flow Cytometry

2 mL platelets (1.0 × 10⁸ mL⁻¹) were seeded in 6-well plates and activated by incubation with 100 µL thrombin (1 U/mL) for at least 20 min. Inactivated platelets or thrombin-activated platelets were incubated with various FITC-labelled tPA-loaded vesicle formulations (equivalent tPA concentration of 0.2 mg mL⁻¹). Free vesicles, which were not attached to platelets, were removed by centrifugation. Data for 1.0 × 10⁴ gated events were collected and analysed using a BD Fortessa II flow cytometer.

### e) Determination of Binding Affinity with Activated Platelets by CLSM

2 mL platelets (1.0 × 10⁸ mL⁻¹) were seeded in 6-well plates, with a collagen-coated glass coverslip on the bottom of each well. After 30 min, 100 µL thrombin (1 U/mL) was added onto the platelet-adhered coverslips to ensure activation of platelets at least 20 min. Inactivated platelets or thrombin-activated platelets were incubated with various FITC-labelled tPA-loaded vesicle formulations (equivalent tPA concentration of 0.2 mg mL⁻¹). The platelets were then fixed with 4.0% formaldehyde for 30 min, followed by rinsing with pH 7.4 PBS buffer for three times. Subsequently, the resulting slides were mounted and observed with a Leica SP₅ MP confocal microscope.

### In Vitro Drug Release Upon Interaction with Activated Platelets

Drug release was evaluated in the presence of activated platelets to confirm the tPA release specifically upon interaction with activated platelets. 200 µL of platelets (1.0 × 10⁸ mL⁻¹) were placed into a collagen-coated 96-well microplate and activated by treatment with 20 µL thrombin (1 µM) for 20 min. Inactivated platelets or thrombin-activated platelets were incubated with various tPA-loaded vesicle formulations (equivalent tPA dose of 0.5 mg mL⁻¹). The released tPA in each well was determined by measuring the absorbance at 405 nm with a GloMax-Multi Microplate Multimode Reader (Promega, USA), as described above in the tPA activity assay. The percentage of tPA release was calculated according to the following equation:

$$\text{tPA} \text{release} \left(\%\right) = \frac{A - {A}_{c}}{{A}_{p} - {A}_{c}} \times 100$$

where A is the absorbance of platelet samples after incubation with the tPA-loaded vesicles; A_{c} is the absorbance of negative control after incubation with the pH 7.4 PBS buffer only; Aₚ is the absorbance of positive control after incubation with the tPA-loaded vesicles lysed by Triton X-100.

### Selective Fibrin Clot Lysis

Fibrin clot lysis by the tPA-loaded vesicles was measured by an agar plate assay. Briefly, 300 mg agar was dissolved in buffer mixture (15 mL of 0.05 M Tris-HCl buffer at pH 7.2 and 5 mL of 0.025 M CaCl₂ solution). 50 mg Fibrinogen was dissolved in 10 mL of Tris-HCl buffer (0.05 M, pH 7.2). The agar solution was mixed with the fibrinogen solution, and then 10 µL thrombin (4.0 µM) was added under stirring for 1 min. The resulting mixture was spread carefully on a transparent plastic plate and homogeneous gels were obtained at 37° C. after 3h. Four sample wells were created in each plate and 5 µL of plasminogen solution (1 mg mL⁻¹) was then added into each sample well. PBS buffer, tPA-RBCVs, tPA-RGD-RBCVs, tPA-cRGD-PEG-RBCVs and free tPA (equivalent tPA dose of 1 mg mL⁻¹) were added into respective sample wells and incubated at 37° C. overnight. The area of the lysed zone in each well was measured to evaluate the degree of fibrin clot lysis.

### Selective Blood Clot Lysis in a Halo Blood Clot Model

Briefly, a clotting mixture (5 mL of buffer containing 66 mM Tris-HCl, 130 mM NaCl and 45 mM CaCl₂; 5 µL of 1 µM thrombin, pH 7.4) was freshly prepared. In a 96-well microplate, 5 µL of this clotting mixture was placed on one side of the well bottom, then 15 µL of whole blood was added on the opposite side of the well bottom. Clotting was initiated by mixing the two drops with a pipette tip in a circular motion to form a homogenous halo shape of blood around the edge of the well bottom, leaving the centre area empty. The plate was covered and incubated at 37° C. for 30 min. After clot formation, 80 µL tPA-RBCVs, tPA-RGD-RBCVs, tPA-cRGD-PEG-RBCVs and free tPA (equivalent tPA dose of 1 mg mL⁻¹) were added simultaneously into respective wells containing halo clots. The dissolution of the halo clots was determined with a plate reader GloMax-Multi Microplate Multimode Reader (Promega, USA) by measuring absorbance at 495 nm caused by RBCs progressively covering the centre of the well after clot degradation at 37° C. The Negative controls for the assay was obtained by adding 80 µL PBS only to halo thrombi (no tPA), and the positive control was obtained by mixing 15 µL blood and 85 µL of PBS in a well (no halo clots). The percentage of clot dissolution was calculated according to the following equation:

$$\% C l o t l y s i s = \frac{A s - {A}_{n}}{{A}_{p} - {A}_{n}} \times 100$$

where Aₛ is the absorbance of well at 495 nm after incubation with samples; Aₙ is the absorbance of negative control well at 495 nm; Aₚ is the absorbance of positive control well at 495 nm.

### Preparation and Characterisation of ASA-Loaded, Fibrinogen-Mimicking, Red Blood Cell-Derived Vesicles (ASA-RGD-RBCVs and ASA-cRGD-PEG-RBCVs)

### a) Preparation of ASA-loaded, Fibrinogen-Mimicking RBCVs (ASA-RGD-RBCVs and ASA-cRGD-PEG-RBCVs)

RBC ghosts were re-suspended in pH 7.4 PBS buffer, which was then added with ASA at a final concentration of 0.5 mg mL⁻¹. DSPE-RGD lipid or DSPE-PEG-cRGD lipid (5.0 × 10⁻⁷ mol) was dissolved in chloroform in a 25-mL round-bottom flask. A lipid film was formed by removal of the organic solvent with rotary evaporation, and then hydrated with 5 mL of ASA-containing RBC ghost solution at 40° C. for 1 h. The mixture solution was sonicated at 4° C. for 30 min, followed by extrusion for times through the 200-nm polycarbonate membrane by using the extruder set. The unencapsulated ASA was removed by dialysis against pH 7.4 PBS solution for 6 h (MWCO = 12 kDa). Meanwhile, ASA-loaded RBCVs without RGD coating (ASA-RBCVs) were synthesised by using the same protocol for comparison.

### b) Vesicle Characterisation

The hydrodynamic size and polydispersity index (PDI) of ASA-cRGD-PEG-RBCVs, ASA-RGD-RBCVs and ASA-RBCVs were measured by DLS (Zetasizer Nano S, Malvern, UK). Their zeta potential was measured by zeta potential analyser (ZetaPALS, Brookhaven, USA).

The absorbance of ASA was measured by UV/Vis spectrophotometry at wavelength of 290 nm. The absorbance value was used to calculate the weight of loaded ASA in vesicles based on the calibration curve obtained for a range of ASA concentrations.

The encapsulation efficiency (EE) was calculated according to the following equation:

$$\text{EE} \left(\%\right) = \frac{{m}_{l}}{{m}_{t}} \times 100$$

where mₗ is the weight of ASA loaded and mₜ is the total weight of ASA in the initial loading solution.

### Binding Affinity with Activated Platelets

### a) Determination of Binding Affinity with Activated Platelets by Flow Cytometry

2 mL platelets (1.0 × 10⁸ mL⁻¹) were seeded in 6-well plates and activated by incubation with 100 µL thrombin (1 U/mL) for at least 20 min. Inactivated platelets or thrombin-activated platelets were incubated with various NBD-labelled RBCV formulations (equivalent NBD-PE of 1.0 × 10⁻⁷ mol). Free vesicles, which were not attached to platelets, were removed by centrifugation. Data for 1.0 × 10⁴ gated events were collected and analysed using a BD Fortessa II flow cytometer.

### c) Determination of Binding Affinity with Activated Platelets by CLSM

2 mL platelets (1.0 × 10⁸ mL⁻¹) were seeded in 6-well plates, with a collagen-coated glass coverslip on the bottom of each well. After 30 min, 100 µL thrombin (1 U/mL) was added onto the platelet-adhered coverslips to ensure activation of platelets for at least 20 min. Inactivated platelets or thrombin-activated platelets were incubated with various NBD-labelled RBCV formulations (equivalent NBD-PE of 1.0 × 10⁻⁷ mol). The platelets were then fixed with 4.0% formaldehyde for 30 min, followed by rinsing with pH 7.4 PBS buffer for three times. Subsequently, the resulting slides were mounted and observed with a Leica SP5 MP confocal microscope.

### In Vitro Antiplatelet Aggregation Assay

The final platelet count was adjusted to 2 × 10⁸ platelets/mL. 150 µL of PRP was seeded in 96-well plates and was incubated with 50 µL of various formulations: ASA-RBCVs, ASA-RGD-RBCVs, ASA-cRGD-PEG-RBCVs or ASA (equivalent ASA concentration of 0.1 mM) in the presence of 10 µL platelet agonist thrombin in PBS (4 µM), arachidonic acid (AA) in PBS (400 µM) or adenosine diphosphate (ADP) in PBS (15 µM). After incubation under shaking at 37° C. for 40 min, the absorbance at 595 nm was measured by a GloMax-Multi Microplate Multimode Reader (Promega, USA). The aggregation percentage was calculated by the equation 2. From the platelet aggregation percentage and comparison to aggregation of PBS without samples, the inhibition percentage was calculated by the equation 3.

### TXB₂ Assay

The procedure of the thromboxane B₂ (TXB₂) ELISA Kit (Abcam, UK) was followed to perform the assay. By using the standard wells of the ELISA plates, a standard curve was obtained to identify the TXB₂ level in the plasma treated with various formulations: PBS, ASA-RBCVs, ASA-RGD-RBCVs, ASA-cRGD-PEG-RBCVs or ASA (equivalent ASA concentration of 0.1 mM).

2 mL of sheep blood was centrifuged at 200 × g for 15 min to collect the plasma. The 1 mL plasma was added in the tube and 500 µL of a specific formulations was then added. The tube was incubated at 37° C. for 10 min, and then 250 µL of arachidonic acid (AA) in PBS (0.2 mg/mL) was added and incubated for another 30 min. The samples were treated according to the procedure of the TXB₂ ELISA Kit, and the absorbance at 405 nm was measured by a GloMax-Multi Microplate Multimode Reader (Promega, USA). The level of TXB₂ was calculated by using the standard curve.

### 6-Keto-PGF_{1α} Assay

The procedure of the 6-keto-PGF_{1α} ELISA Kit (Abcam, UK) was followed to perform the assay. By using the standard wells of the ELISA plates, a standard curve was obtained to identify the 6-keto-PGF_{1α} level in the plasma treated with various formulations: PBS, ASA-RBCVs, ASA-RGD-RBCVs, ASA-cRGD-PEG-RBCVs or ASA (equivalent ASA concentration of 0.1 mM).

2 mL of sheep blood was centrifuged at 200 × g for 15 min to collect the plasma. The 1 mL plasma was added in the tube and 500 µL of a specific formulation was then added. The tube was incubated at 37° C. for 10 min, and then 250 µL of arachidonic acid (AA) in PBS (0.2 mg/mL) was added and incubated for another 30 min. The samples were treated according to the procedure of the 6-keto-PGF_{1α} ELISA Kit, and the absorbance at 405 nm was measured by a GloMax-Multi Microplate Multimode Reader (Promega, USA). The level of 6-keto-PGF_{1α} was calculated by using the standard curve.

### In Vitro Antithrombotic Assay

200 µL of sheep blood was added into a weighted tube. Then, 50 µL specific formulation (PBS, ASA-RBCVs, ASA-RGD-RBCVs, ASA-cRGD-PEG-RBCVs or ASA at an equivalent ASA concentration of 0.1 mM) was added in the tube, and then 10 µL of thrombin in PBS (4 µM) was added in each well and incubated at 37° C. under shaking for 30 min. Finally, the un-clotted blood was removed to obtain the weight of the wet clotted thrombus.

### Example 1 - Red Blood Cell-Derived Vesicles (RBCVs) for Targeted Thrombolysis

The inventors set out to create a novel delivery means for a thrombolytic agent, such as tPA. Inspired from the fibrinogen binding with activated platelets at a thrombus site, as shown in FIG. 1 (top left), the inventors developed novel fibrinogen-mimicking systems based on surface coating of natural red blood cell-derived vesicles (RBCVs) with peptides containing RGD sequences. The fibrinogen-mimicking RBCVs (FM-RBCVs) can be encapsulated with thrombolytic agents, including tPA, the resulting RBCVs having high selectivity and affinity towards activated platelets for targeted delivery of tPA to a thrombus site and triggered release locally for effective thrombolysis without minimised off-target bleeding side effects.

### Results & Discussion

The inventors made novel red blood cell derived vesicles (RBCV) 1, as shown in FIG. 1 (top right), where the RBCV (1) comprises an outer lipid bilayer membrane (6), derived from a red blood cell (7) defining an intravesicular space (8), the RBCV (1) being decorated with a plurality of spaced apart platelet-targeting moieties or ligands (2), such as RGD and cRGD. The RBCVs are able to mimic the action of fibrinogen (9) by the presence of the RGD ligands (2), which functionalise the surface of the RBCV (1). Fibrinogen (9) is capable of binding to GPIIb-IIIa (α_{IIb}β₃) integrin (10) on activated platelets (12) through the arginine-glycine-aspartic acid (RGD) motifs located on the two Aα chains (14). This leads to platelet aggregation (13) through the “bridging effect”, which leads to thrombus (5) formation and the occlusion of a blood vessel (4), as shown in FIG. 1 (top left and bottom left). Thus, RBCVs (1) comprising an RGD ligand (2) are capable of binding to activated platelets (12) to enable targeted delivery of the active agent (3) contained within the intravesicular space, for example tPA (3), to activated platelets (12) in the thrombus (5) present in a blood vessel (4), as shown in FIG. 1 (bottom left). After delivery of the active agent (3) to the thrombus (5), the agent may act to lyse the clot (15), removing the thrombus (5), thus enabling the flow of blood cells (7) along the blood vessel (4), as shown in FIG. 1 bottom left.

The inventors also devised an elegant method to produce RBCVs for targeted thrombus delivery, as shown in FIG. 2. Red blood cells (7) were treated with a hypotonic solution (16) comprising EDTA (17) to substantially remove haemoglobin from the red blood cells (7). After steps of centrifugation, removing the supernatant and repeating EDTA treatment until the solution is free of red colour, the resulting red blood cell ghosts (19) were suspended in a PBS solution (18). tPA (3) was added to the solution and this was added to a dehydrated DSPE-RGD or DSPE-PEG-cRGD lipid (20). The solution was sonicated (21) and then extruded (22) through a 200 nm polycarbonate membrane to produce a filtrate, and unencapsulated tPA was removed by ultracentrifugation of the filtrate producing the tPA (3)-loaded, fibrinogen-mimicking RBCVs (1) (tPA-RGD-RBCVs or tPA-cRGD-PEG-RBCVs).

### Platelet Targeting Moiety

Activation of the GPIIb-IIIa (α_{IIb}β₃) integrin is the common pathway involved in platelet aggregation. Normally, α_{IIb}β₃ integrins are inactive on the circulating resting platelet surface. However, in the event of thrombus formation, platelets will be in an active state α_{IIb}β₃ integrins are abundantly expressed and activated with a conformational change on the platelet membrane. This conformational change of α_{IIb}β₃ integrins allows specific binding of activated platelets to fibrinogens through the arginine-glycine-aspartic acid (RGD) motifs located in each of its two Aα chains, which leads to platelet aggregation through the “bridging effect”. Therefore, activated platelets are an ideal target for the selective delivery of thrombolytic agents to thrombi, because the aggregation of activated platelets and abundant active α_{IIb}β₃ integrins on the surface of activated platelets are the significant hallmark events in thrombosis.

One embodiment of RBCVs made is referred to tPA-RBCVs - this includes a red blood cell-derived vesicle (1) encapsulating the active agent, tPA (3).

The second embodiment is known as tPA-RGD-RBCVs - this includes a red blood cell-derived vesicle (1) decorated with a plurality of RGD ligands and encapsulating the active agent, tPA (3).

The third embodiment is known as tPA-cRGD-PEG-RBCVs - this includes a red blood cell-derived vesicle (1) decorated with a plurality of cRGD ligands and encapsulating the active agent, tPA (3).

Table 1

Mean hydrodynamic sizes measured by dynamic light scattering (DLS), encapsulation efficiencies and zeta potentials of tPA-RBCVs, tPA-RGD-RBCVs and tPA-cRGD-PEG-RBCVs

| Vesicles | DLS size (nm) | DLS size after 4 weeks (nm) | Encapsulation efficiency (%) | Zeta potential (mV) |
|---|---|---|---|---|
| tPA-RBCVs | 265.7±5.1 | 395.1±6.7 | 24.3±3.2 | -30.1±3.9 |
| tPA-RGD-RBCVs | 253.7±2.4 | 282.5±4.3 | 27.9±2.8 | -27.9±4.5 |
| tPA-cRGD-PEG-RBCVs | 260.3±3.8 | 265.1±3.4 | 29.2±2.5 | -35.8±3.7 |

As shown in Table 1 and FIG. 3, the DLS sizes of tPA-RGD-RBCVs (253.7 ± 2.4 nm) and tPA-cRGD-PEG-RBCVs (260.3±3.8 nm) were similar to that of tPA-RBCVs (265.7±5.1 nm). This suggests that RGD or cRGD conjugation onto the vesicle surface did not cause a significant change in particle size. There was no significant size change for tPA-RGD-RBCVs or tPA-cRGD-PEG-RBCVs after 4 weeks of storage at 4° C., but a considerable size increase was detected for tPA-RBCVs. The TEM image confirmed that tPA-RGD-RBCVs were spherical in shape (see FIG. 4) and that the TEM particle size was consistent with the DLS result. The encapsulation efficiencies of tPA-RGD-RBCVs (27.9 ± 2.8%) and tPA-cRGD-PEG-RBCVs (29.2±2.5%) were similar to that for tPA-RBCVs (24.3 ± 3.2%). Zeta potential values of the tPA-RBCVs, tPA-RGD-RBCVs and tPA-cRGD-PEG-RBCVs were in the range of -35.8±3.7 to -27.9 ± 4.5 mV.

FIG. 5 shows the concentration-dependent effect of free peptides (RGD and cRGD) on the inhibition of platelet aggregation. Free cRGD caused 50% inhibition of platelet aggregation at a much lower peptide concentration (0.46±2.5 mM) compared to free linear RGD (381.7±6.3 mM). This confirms that both the α_{IIb}β₃-specific peptides (linear RGD and cRGD) can kinetically outcompete with natural ligand fibrinogen in binding to active α_{IIb}β₃ and hence prevent fibrinogen-mediated platelet aggregation. The higher affinity peptide can outcompete fibrinogen at low peptide concentrations while the lower affinity peptide requires much higher concentrations to gain the kinetic advantage. Hence, affinity was directly correlated with the IC₅₀ value and the value for cRGD was found to be considerably lower than that for linear RGD.

As shown in FIG. 6A, resting platelets treated with FITC-labelled tPA-RBCVs, tPA-RGD-RBCVs and tPA-cRGD-PEG-RBCVs displayed a similar level of fluorescence intensity, which showed only a small increase compared to that of the control resting platelets treated with pH 7.4 PBS buffer alone (FIG. 6B). This suggests that the three vesicle formulations had a low level of attachment to resting platelets.

FIG. 7A shows that, even when platelets were activated by thrombin, only a slight increase in fluorescence intensity was observed for activated platelets treated with FITC-labelled tPA-RBCVs. However, FITC-labelled tPA-RGD-RBCVs and tPA-cRGD-PEG-RBCVs bound more avidly to activated platelets. FIG. 7B shows that the fluorescence intensity in activated platelets after treated with FITC-labelled tPA-RGD-RBCVs was a little lower than the treatment with FITC-labelled tPA-cRGD-PEG-RBCVs, but had an over 4-fold increase as compared to the treatment with FITC-labelled tPA-RBCVs. These results suggest that RGD peptides, especially cRGD peptides, can efficiently facilitate the specific binding of the tPA-loaded fibrinogen-mimicking RBC-derived vesicles to activated platelets.

As shown in FIG. 8A, the resting platelets treated with FITC-labelled tPA-RBCVs, tPA-RGD-RBCVs and tPA-cRGD-PEG-RBCVs only showed very weak green fluorescence. However, tPA-RGD-RBCVs and tPA-cRGD-PEG-RBCVs displayed significantly enhanced staining of activated platelets as compared to FITC-labelled tPA-RBCVs without RGD peptide coating. Analysis of the MFI in those images (FIG. 8B) suggested that tPA-RGD-RBCVs had a little lower binding affinity to activated platelets than tPA-cRGD-PEG-RBCVs, but showed an approximately 4-fold enhancement in binding affinity to activated platelets as compared to tPA-RBCVs. This is in good agreement with the flow cytometry results shown in FIG. 7B, further consolidating the ability of tPA-RGD-RBCVs and tPA-cRGD-PEG-RBCVs to efficiently facilitate targeted drug delivery to activated platelets at a thrombus site.

The amount of RGD arms can be controlled to maximise the binding affinity. As shown in FIG. 9, there was a sharp increase in the binding affinity of tPA-RGD-RBCVs to activated platelets when the RGD arm density increased from 1.0 × 10⁴ to 4.0 × 10⁴ arms per vesicle, beyond which the influence of RGD arm coating density was insignificant. This suggests that 4.0 × 10⁴ arms per vesicle was an important threshold value for RGD to achieve maximal targeting to activated platelets.

As shown in FIG. 10, upon binding of tPA-RGD-RBCVs or tPA-cRGD-PEG-RBCVs to activated platelets, around 70% of the entrapped tPA was released within 2 h, and the release of tPA continued to increase to about 85% in 6 h, which was considerably higher than the tPA release from tPA-RBCVs (about 30% within 6 h). By comparison, when incubated with resting platelets, tPA-RGD-RBCVs released only around 30% of the entrapped tPA in 6 h. These results indicate that drug release from the fibrinogen-mimicking vesicles was preferably induced by the interaction of RGD peptides on the surface of tPA-RGD-RBCVs with the α_{IIb}β₃ on the surface of activated platelets.

A fluorescence resonance energy transfer (FRET) assay was carried out to examine if the fibrinogen-mimicking RBCVs released drug payload via lipid membrane destabilisation involving membrane fusion between vesicles and activated platelets. As shown in FIG. 11, the NBD fluorescence was enhanced rapidly and considerably upon incubation of the RGD-RBCVs or cRGD-PEG-RBCVs nanovesicles with activated platelets. This was attributed to the decreased surface density of the donor NBD and the increased distance between the donor NBD and the acceptor Rhod resulting from the fusion between the RBCV membrane and the activated platelet membrane. By contrast, incubation RGD-RBCVs with resting platelets caused minimal membrane fusion and a consequently negligible increase in the NBD fluorescence. Inhibition of α_{IIb}β₃ integrins on activated platelets with eptifibatide caused a negligible increase in the NBD fluorescence, confirming that this membrane fusion was induced by specific interaction between RGD peptide arms and α_{IIb}β₃ integrins.

The fibrinolytic activity of tPA-RGD-RBCVs and tPA-cRGD-PEG-RBCVs was evaluated in the presence of activated platelets to confirm the selective fibrin lysis. As shown in FIG. 12A, no significant change of the fibrin clot was observed after incubation with PBS buffer, suggesting that PBS buffer did not break up the fibrin clot. Only a very small lysis ring was observed after incubation with tPA-RBCVs, which might be due to the marginal tPA release from tPA-RBCVs. Interestingly, upon treatment of the fibrin clot with tPA-RGD-RBCVs or tPA-cRGD-PEG-RBCVs, a clear clot lysis zone around the sample well was observed, showing a similar size of the lysis zone caused by free tPA. The areas of the fibrin clot lysis rings were calculated in FIG. 12B. It was shown that, in the presence of activated platelets, tPA-RGD-RBCVs and tPA-cRGD-PEG-RBCVs caused significant fibrin lysis and the areas of lysis ring were 1.13 ± 0.12 cm² and 1.15 ± 0.18 cm², respectively, which were similar to that of free tPA (1.18 ± 0.17 cm²) but significantly higher than that of tPA-RBCVs (0.14 ± 0.07 cm²) and PBS buffer (0.02 ± 0.01 cm²) . These results suggest that the tPA-loaded fibrinogen-mimicking vesicles can cause efficient fibrin lysis in the presence of activated platelets.

The representative photographs of Halo blood clots after treatment with PBS buffer, blank RBCVs, tPA-RBCVs, tPA-RGD-RBCVs, tPA-cRGD-PEG-RBCVs and free tPA, respectively are displayed in FIG. 13A. It was clearly found that the clots were dissolved and consequently RBCs were released after incubation with tPA-RGD-RBCVs or tPA-cRGD-PEG-RBCVs, similar to the clots treated with free tPA. By contrast, the remnant clots were visible to the naked eye after treatment with tPA-RBCVs, blank RBCVs or PBS buffer. As shown in FIG. 13B, the clot-lytic activity of tPA-RGD-RBCVs or tPA-cRGD-PEG-RBCVs was a bit lower than free tPA at the initial timepoints, which could be ascribed to the time required for tPA release from the fibrinogen-mimicking vesicles. However, after 45 min of treatment, both tPA-RGD-RBCVs and tPA-cRGD-PEG-RBCVs, like free tPA, caused the complete blood clot lysis. By comparison, the degree of blood clot dissolution by tPA-RBCVs (without RGD coating) was found to be about 50%, but the degree of blood clot lysis for blank RBCVs and PBS buffer were both less than 5% after incubation for 45 min. All these results demonstrated that these tPA-loaded fibrinogen-mimicking vesicles had considerably higher thrombolytic activity compared to the non RGD-coated vesicles.

### Example 2 - Red Blood Cell-Derived Vesicles (RBCVs) for Antithrombotic Study

The inventors set out to create a novel delivery means for an anti-platelet agent, such as acetylsalicylic acid (ASA, aspirin), by using the fibrinogen-mimicking RBCVs. The fibrinogen-mimicking RBCVs can be encapsulated with anti-platelet agents, including ASA. The resulting ASA-loaded, fibrinogen-mimicking RBCVs have high selectivity and affinity towards activated platelets for targeted delivery and triggered release of ASA for effective antithrombotic effect.

### Results & Discussion

The inventors used the same method, as described above, to produce fibrinogen-mimicking RBCVs and encapsulate ASA (FIGS. 14 and 15) for antithrombotic study as mentioned above.

One embodiment of RBCVs is known as ASA-RBCVs - this includes a red blood cell-derived vesicle encapsulating the active agent, ASA.

The second embodiment is known as ASA-RGD-RBCVs - this includes a red blood cell-derived vesicle decorated with a plurality of RGD ligands and encapsulating the active agent, ASA.

The third embodiment is known as ASA-cRGD-PEG-RBCVs - this includes a red blood cell-derived vesicle decorated with a plurality of cRGD ligands and encapsulating the active agent, ASA.

Table 2

Mean hydrodynamic sizes measured by dynamic light scattering (DLS), encapsulation efficiencies and zeta potentials of ASA-RBCVs, ASA-RGD-RBCVs and ASA-cRGD-PEG-RBCVs

| Vesicles | DLS size (nm) | DLS size after 4 weeks (nm) | Encapsulation efficiency (%) | Zeta potential (mV) |
|---|---|---|---|---|
| ASA-RBCVs | 255.8±4.9 | 416.1±7.1 | 16.9±5.1 | -20.1±5.4 |
| ASA-RGD-RBCVs | 256.6±2.7 | 281.8±5.9 | 17.7±4.8 | -22.7±3.2 |
| ASA-cRGD-PEG-RBCVs | 258.1±4.2 | 264.9±3.1 | 21.4±3.7 | -29.3±4.4 |

As shown in Table 2, the DLS size of ASA-RGD-RBCVs (256.6±2.7 nm) and ASA-cRGD-PEG-RBCVs (258.1±4.2 nm) were similar to that of ASA-RBCVs (255.8±4.9 nm). This suggests that RGD or cRGD conjugation onto the vesicle surface did not cause a significant change in particle size. There was no significant size change for ASA-RGD-RBCVs and ASA-cRGD-PEG-RBCVs after 4 weeks of storage at 4° C., but a considerable size increase was detected for ASA-RBCVs. The encapsulation efficiency of the ASA-RBCVs, ASA-RGD-RBCVs and ASA-cRGD-PEG-RBCVs were in the range of 16.9±5.1% to 21.4±3.7%. Zeta potential values of the ASA-RBCVs, ASA-RGD-RBCVs and ASA-cRGD-PEG-RBCVs were in the range of -29.3±4.4 to -20.1±5.4 mV.

As shown in FIG. 16A, resting platelets treated with NBD-labelled RBCVs, RGD-RBCVs and cRGD-PEG-RBCVs displayed a similar level of fluorescence intensity, which showed only a small increase compared to that of the control resting platelets treated with pH 7.4 PBS buffer alone (FIG. 16B). This suggests that the three vesicle formulations had a low level of attachment to resting platelets.

FIG. 17A shows that, even when platelets were activated by thrombin, only a slight increase in fluorescence intensity was observed for activated platelets treated with NBD-labelled RBCVs. However, NBD-labelled RGD-RBCVs and cRGD-PEG-RBCVs bound more avidly to activated platelets. FIG. 17B shows that the fluorescence intensity in activated platelets after treated with NBD-labelled RGD-RBCVs was a little lower than the treatment with NBD-labelled cRGD-PEG-RBCVs, but had an over 4-fold increase as compared to the treatment with NBD-labelled RBCVs. These results suggest that RGD peptides, especially cRGD peptides, can efficiently facilitate the specific binding of the fibrinogen-mimicking RBC-derived vesicles to activated platelets.

As shown in FIG. 18A, the resting platelets treated with NBD-labelled RBCVs, RGD-RBCVs and cRGD-PEG-RBCVs only showed very weak green fluorescence. However, RGD-RBCVs and cRGD-PEG-RBCVs displayed significantly enhanced staining of activated platelets as compared to RBCVs without RGD peptide coating. Analysis of the MFI in those images (FIG. 18B) suggested that RGD-RBCVs had a slightly lower binding affinity to activated platelets than that of cRGD-PEG-RBCVs, but showed an approximately 4-fold enhancement in binding affinity to activated platelets as compared to RBCVs. This is in good agreement with the flow cytometry results shown in FIG. 17B, further consolidating the ability of RGD-RBCVs and cRGD-PEG-RBCVs to efficiently facilitate binding to activated platelets.

The in vitro activities of ASA-RGD-RBCVs and ASA-cRGD-PEG-RBCVs inhibiting adenosine diphosphate (ADP)-, arachidonic acid (AA)- or thrombin-induced platelet aggregation were determined. As shown in FIG. 19A, the percentages of inhibition of ADP-induced platelet aggregation by ASA-RBCVs, ASA-RGD-RBCVs, ASA-cRGD-PEG-RBCVs and ASA (equivalent ASA concentration of 0.1 mM) were 16.31±2.48%, 21.76±5.19%, 29.41±3.35% and 27.56±2.71%, respectively. This suggests that for ADP-induced platelet aggregation ASA-RGD-RBCVs and ASA-cRGD-PEG-RBCVs displayed a similar level of inhibition to that of free ASA, but higher than that of ASA-RBCVs without RGD. As shown in FIG. 19B, the percentages of inhibition of AA-induced platelet aggregation by ASA-RBCVs, ASA-RGD-RBCVs, ASA-cRGD-PEG-RBCVs and ASA (equivalent ASA concentration of 0.1 mM) were 11.36±4.57%, 22.41±3.42%, 23.89±2.43% and 23.81±4.26%, respectively. This suggests that for AA-induced platelet aggregation ASA-RGD-RBCVs and ASA-cRGD-PEG-RBCVs displayed a similar level of inhibition to that of free ASA, but higher than that of ASA-RBCVs without RGD. As shown in FIG. 19C, the percentages of inhibition of thrombin-induced platelet aggregation by ASA-RBCVs, ASA-RGD-RBCVs, ASA-cRGD-PEG-RBCVs and ASA (equivalent ASA concentration of 0.1 mM) were 12.24±3.41%, 13.71±3.31%, 21.84±3.22% and 20.47±1.04%, respectively. This indicates that for thrombin-induced platelet aggregation ASA-cRGD-PEG-RBCVs displayed a similar level of inhibition to that of free ASA, but higher than that of ASA-RBCVs and ASA-RGD-RBCVs.

ASA's ability to suppress the production of prostaglandins and thromboxanes is attributed to its irreversible inactivation of the cyclooxygenase (COX) enzyme (FIG. 20), which results in the prevention of clotting. To evidence the inhibition of COX, the levels of thromboxane B2 (TXB2) and 6-keto-PGF1α were measured according to the manufacturer's guidance using the TXB2 ELISA kit and 6-keto-PGF1α ELISA kit (Abcam), respectively. As shown in FIGS. 21A and 21B, ASA-RGD-RBCVs and ASA-cRGD-PEG-RBCVs significantly decreased the levels of both TXB2 and 6-keto-PGF1α as compared to ASA-RBCVs without RGD, confirming the efficient inhibition of the COX-mediated AA metabolism by the ASA-loaded fibrinogen-mimicking RBC-derived vesicles.

The in vitro antithrombotic activities of ASA-RBCVs, ASA-RGD-RBCVs, ASA-cRGD-PEG-RBCVs and ASA were evaluated by comparing the thrombus weight. As shown in FIGS. 22A and 22B, ASA-RBCVs, ASA-RGD-RBCVs, ASA-cRGD-PEG-RBCVs and ASA exhibited varied antithrombotic activities, with the thrombus weight of 94.60±4.69, 75.61±9.01, 53.87±8.52 and 49.27±10.73 mg, respectively. This indicates that ASA-loaded fibrinogen-mimicking RBC-derived vesicles showed the significantly higher antithrombotic efficiency than that of ASA-RBCVs without RGD. The high inhibition of clotting could result from the selective delivery and the consequent triggered release of ASA due to the presence of RGD for ASA-RGD-RBCVs and the presence of cRGD for ASA-cRGD-PEG-RBCVs. The stronger binding affinity of cRGD suggested a higher antithrombotic ability of ASA-cRGD-PEG-RBCVs than ASA-RGD-RBCVs.

### Conclusions

The inventors have, for the first time, described the use of RGD-coated RBCVs (linear and cyclic) as activated-platelet-sensitive nanocarriers for thrombus-targeted delivery of thrombolytic drugs, including tPA, for safer and more effective thrombolytic therapy, or as anti-platelet agents that decrease platelet aggregation and inhibit thrombus formation.

The naturally derived, camouflaged nanovesicles are biocompatible, biodegradable and non-immunogenic, and can achieve a very long circulation in the bloodstream. The RGD on the RBCV surface can enable a very high selectivity and affinity binding with the α_{IIb}β₃ integrin overexpressed on activated platelets at a blood clot site, thus leading to the efficient, triggered release of thrombolytic agents locally and the consequent targeted thrombolysis.

In summary, therefore, the RBCVs enable selective delivery of thrombolytics to a blood clot site and controlled release locally. This targeted delivery enhances clot disrupting efficacy, limit drug dose and attenuate off-target bleeding side effects. In addition, the RBCVs enable selective delivery of anti-platelet agents to activated platelets and controlled release locally, which leads the inhibition of platelet aggregation, enhanced antithrombotic efficacy, and reduced drug dose. The RBCV enables more patients with thrombotic diseases to receive safer and more effective treatment. In addition, the RBCVs provide a support surface for anchorage of controllable amounts of RGDs such as linear RGD or cRGD. The resulting multi-arm nanovesicles have superior selectivity and strong binding with activated platelets.

The use of the RBCVs provide for a triggered release mechanism. Before reaching a thrombus, RBCVs protect thrombolytic agents in the blood circulation, leading to considerably improved stability and prolonged half-life and temporarily suppress thrombolytic activity, leading to reduced haemorrhagic side effects. Upon selective binding to activated platelets, the RBCVs fuse with the activated platelet membrane, leading to rapid and efficient release of thrombolytics. This is favourable for treatment of acute events including but not limited to ischemic stroke which requires immediate drug action. It may also be favourable for lysis of both fibrin-rich (responsive to tPA) and platelet-rich blood clots (resistant to tPA), thus with broader potential clinical applications.

The novel RBCVs advantageously:
(i) encapsulate and protect drugs in the bloodstream with considerably improved stability and elongated half-life,
(ii) temporarily suppress thrombolytic activity of thrombolytics in the bloodstream and thus reduce the risk of systemic bleeding,
(iii) target thrombolytic drugs to blood clots and thus improve its efficacy without increasing off-targets,
(iv) selectively bind to activated platelets, which can cause efficient and rapid controlled drug release locally as a result of membrane fusion; and
(v) enhance penetration of drugs into clots and thus lead to efficient recanalisation;
(vi) enable targeted delivery of antiplatelet drugs to activated platelets, which can cause efficient and rapid controlled drug release locally, thus decreasing platelet aggregation and inhibiting thrombus formation.

1. A red blood cell-derived vesicle comprising an encapsulated active agent.

2. The vesicle according to claim 1, comprising at least one targeting ligand or moiety attached to the surface thereof.

3. The vesicle according to claim 2, wherein the at least one targeting ligand or moiety is configured to selectively target the vesicle to a cell involved in blood clotting or a factor present in the characteristic microenvironment of a thrombus site.

4. The vesicle according claim 2 , wherein the at least one targeting ligand or moiety is configured to selectively target the vesicle to a platelet.

5. The vesicle according to claim 2, wherein the at least one targeting ligand is a peptide selected from a group of peptides consisting of: SEQ ID No: 1; SEQ ID No: 2; SEQ ID No: 3; SEQ ID No: 4; SEQ ID No: 5; SEQ ID No: 6; SEQ ID No: 7; SEQ ID No: 8; SEQ ID No: 9; SEQ ID No: 10; SEQ ID No: 11; SEQ ID No: 12; SEQ ID No: 13; SEQ ID No: 14; SEQ ID No: 15; and SEQ ID No: 16.

6. (canceled)

7. The vesicle according to claim 1, wherein the active agent is a thrombolytic agent.

8. The vesicle according to claim 1, wherein the active agent is selected from a group consisting of: a fibrinolytic agent; a von Willebrand factor-cleaving protease (VWFCP); and a DNase that is capable of degrading neutrophil extracellular traps (NETs).

9. The vesicle according to claim 7, wherein the thrombolytic agent is tPA.

10. The vesicle according to claim 1, wherein the active agent is an antiplatelet agent selected from a group consisting of: a GPIIb-IIIa α_{IIb}β₃) inhibitor; an irreversible cyclooxygenase inhibitor; an adenosine diphosphate (ADP) receptor inhibitor; an adenosine reuptake inhibitor; a phosphodiesterase inhibitor; a protease-activated receptor-1 (PAR-1) antagonist; and a thromboxane inhibitor.

11. The vesicle according to claim 10, wherein the anti-platelet agent is acetylsalicylic acid (aspirin, ASA).

12. The vesicle according to 6 claim 1, comprising an active agent selected from a thrombolytic agent, a fibrinolytic agent, a von Willebrand factor-cleaving protease (VWFCP); and a DNase that is capable of degrading neutrophil extracellular traps (NETs) comprising an anti-platelet agent selected from a group consisting of: a GPIIb-IIIa (a_{IIb}β₃) inhibitor; an irreversible cyclooxygenase inhibitor; an adenosine diphosphate (ADP) receptor inhibitor; an adenosine reuptake inhibitor; a phosphodiesterase inhibitor; a protease-activated receptor-1 (PAR-1) antagonist; a thromboxane inhibitor; and acetylsalicylic acid (aspirin, ASA).

13. A method of preparing a red blood cell-derived vesicle (RBCV) comprising an encapsulated active agent, the method comprising:
(i) contacting a red blood cell with a hypotonic solution to produce a red blood cell ghost; and
(ii) encapsulating an active agent using the red blood cell ghost, to thereby produce a red blood cell-derived vesicle comprising an encapsulated active agent.

14. The method according to claim 13, wherein the active agent is as defined in claim 7.

15. The method according to claim 13 , wherein step (ii) further comprises contacting the red blood cell ghost with at least one targeting ligand or targeting moiety, and wherein the at least one targeting ligand or targeting moiety is configured to selectively target the vesicle to a cell involved in blood clotting or a factor present in the characteristic microenvironment of a thrombus site.

16-25. (canceled)

26. A method of treating, preventing, ameliorating, or reducing a thrombotic disorder or a blood clot, the method comprising administering, or having administered, to a subject in need thereof, a therapeutic amount of the red blood cell vesicle according to claim 1.

27. The method of claim 26, wherein the thrombotic disorder is selected from the group consisting of: ischemic stroke; myocardial infarction and pulmonary embolism.
